SEQUENCE LISTING (44 sequences)

SEQ ID NO: 1 - protein, 246 aa, Momordica charantia
DVSFRLSGADPRSYGMFIKDLRNALPFREKVYNIPLLLPSVSGAGRYLLMHLFNYDGKTITVAVDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVTSKVVTSNIQLLLNTRNI

SEQ ID NO: 2 - protein, 247 aa, Bryonia dioica
DVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLENNWSALSKQIQIASTNNGQFESPVVLIDGNNQRVSITNASARVVTSNIALLLNRNNIA

SEQ ID NO: 3 - protein, 244 aa, Cucurbita moschata
NVRFDLSSATSSSYKTFIKNLREALPKDGKVYDIPVLLSTVMDSRRFILIDLVNYDGQSITAAIDVLNVYIVAYSTGTVSYFFQQVPAQAPKLLFKGTQQRTLPYTGNYENLQTAAKKLRENIELGLPALDSAITTLFHYNAEAAASALLVLIQTTSEAARFRYIELQIANNVGTKFKPSQTIISLENNWSALSKQIQIAKNKNGQFETPVILIDPQGNRVQITNVTSNVVTQNIQLLLNIGAT

SEQ ID NO: 4 - protein, 261 aa, Bryonia dioica
VDINFSLIGATGATYKTFIRNLRTKLTVGTPRVYDIPVLRNAAAGLARFQLVTLTNYNGESVTVALDVVNVYVVAYRAGNTAYFLADASTEANNVLFAGINHVRLPYGGNYDGLETAAGRISRENIELGFSEISSAIGNMFRHNPGTSVPRAFIVIIQTVSEAARFKYIEQRVSENVGTKFKPDPAFLSLQNAWGSLSEQIQIAQTRGGEFARPVELRTVSNTPTFVTNVNSPVVKGIALLLYFRVNVGTDNVFAMSLSTY

SEQ ID NO: 5 - protein, 247 aa, Trichosanthes kirilowii
DVSFRLSGATSSSYGVFISNLRKALPNERKLYDIPLLRSSLPGSQRYALIHLTNYADETISVAIDVTNVYIMGYRAGDTSYFFNEASATEAAKYVFKDAMRKVTLPYSGNYERLQTAAGKIRENIPLGLPALDSAITTLFYYNANSAASALMVLIQSTSEAARYKFIEQQIGKRVDKTFLPSLAIISLENSWSALSKQIQIASTNNGQFESPVVLINAQNQRVTITNVDAGVVTSNIALLLNRNNMA

SEQ ID NO: 6 - protein, 249 aa, Trichosanthes kirilowii
EGDVSFRLSGATSSSYGVFISNLRKALPYERKLYDIPLLRSTLPGSQRYALIHLTNYADETISVAIDVTNVYVMGYRAGDTSYFFNEASATEAAKYVFKDAKRKVTLPYSGNYERLQIAAGKIRENIPLGLPALDSAITTLFYYNANSAASALMVLIQSTSEAARYKFIEQQIGKRVDKTFLPSLAIISLENSWSALSKQIQIASTNNGQFETPVVLINAQNQRVTITNVDAGVVTSNIALLLNRNNMA

SEQ ID NO: 7 - protein, 275 aa, synthetic construct
VEGDVSFRLLGATTKSYKDFIANFRNDLASHKRVYNIRLLSLTVPGPSRYTLVHLYNYDDQTITLAVDVINVYIMGYRNGHVSYFFNEPAAAEASKFVFKDAQRRVTLPFTSNYAGLETAAGKIRERIPLGLPAMNNAITNLVYYDTSPRNAIAAAFLVLIQSTAEAARFQYIEKVIGKDVEKDFLPSEALLSLENNWGALSKQIQIAEKNNGKFKDPVLLTDNQGKKVQINNVGSNVVTANIQLLLNYQQQVLAFDQDIMSSELRLGHGSYVTM

SEQ ID NO: 8 - protein, 283 aa, Mirabilis expansa
APPTLATLDLTAAANYPPFITNMRNVLSEKDKNGKDVLLCTMKKISTTVPSPRYAYVDIKASATQTVTLAIDRTNTYVLGYRDIFGGTDRAAFFKDVYDDAKDLFPDAKGKNRIKLSYGSQYTTLGDRTKVPLGIKSLRISITAIYGEAAGTDLDKNRREFFLLALQMVAEATRFKYISDKIPTERDYDTLKVDNHMIALENGWDLLSTAIYNAKSSTTKPTKCELLKTPVSLIWLGQNEWNFNSVEEIAKVVGLLKAKGKKLSTNNDDDNNGDDCGSVVVAS

SEQ ID NO: 9 - protein, 247 aa, synthetic construct
DVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLENNWSALSKQIQIASTNNGQFESPVVLIDGNNQRVSITNADARVVTSNIALLLNRNNIA

SEQ ID NO: 10 - protein, 247 aa, synthetic construct; identical to SEQ ID NO: 9

SEQ ID NO: 11 - protein, 247 aa, synthetic construct
DVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLENSWSALSKQIQIASTNNGQFESPVVLIDGNNQRVSITNADARVVTSNIALLLNRNNIA

SEQ ID NO: 12 - protein, 246 aa, synthetic construct
DVSFRLSGADPRSYGMFIKDLRNALPFREKVYNIPLLLPSVSGAGRYLLMHLFNYDGKTITVAVDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVGSKVVTSNIQLLLNTRNI

SEQ ID NO: 13 - protein, 246 aa, synthetic construct
DVSFRLSGADPRSYGMFIKDLRNALPFREKVYNIPLLLPSVSGAGRYLLMHLFNYDGKTITVAVDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVDSKVVTSNIQLLLNTRNI

SEQ ID NO: 14 - protein, 244 aa, synthetic construct
NVRFDLSSATSSSYKTFIKNLREALPKDGKVYDIPVLLSTVMDSRRFILIDLVNYDGQSITAAIDVLNVYIVAYSTGTVSYFFQQVPAQAPKLLFKGTQQRTLPYTGNYENLQTAAKKLRENIELGLPALDSAITTLFHYNAEAAASALLVLIQTTSEAARFRYIELQIANNVGTKFKPSQTIISLENSWSALSKQIQIAKNKNGQFETPVILIDPQGNRVQITNVGSNVVTQNIQLLLNIGAT

SEQ ID NO: 15 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 698 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDVSFRLSGATTTSYGVFIKNLREALPYERKVYNIPLLRSSISGSGRYTLLHLTNYADETISVAVDVTNVYIMGYLAGDVSYFFNEASATEAAKFVFKDAKKKVTLPYSGNYERLQTAAGKIRENIPLGLPALDSAITTLYYYTASSAASALLVLIQSTAESARYKFIEQQIGKRVDKTFLPSLATISLENNWSALSKQIQIASTNNGQFESPVVLIDGNNQRVSITNASARVVTSNIALLLNRNNIA

SEQ ID NO: 18 - protein, 9 aa, synthetic construct
YTFTSYNMH

SEQ ID NO: 19 - protein, 14 aa, synthetic construct
WIGAIYPGNGDTSY

SEQ ID NO: 20 - protein, 13 aa, synthetic construct
RSTYYGGDWYFNV

SEQ ID NO: 21 - protein, 7 aa, synthetic construct
SSVSYIH

SEQ ID NO: 22 - protein, 11 aa, synthetic construct
PWIYATSNLAS

SEQ ID NO: 23 - protein, 8 aa, synthetic construct
QQWTSNPP

SEQ ID NO: 24 - protein, 9 aa, synthetic construct
EEQYNSTYR

SEQ ID NO: 25 - protein, 13 aa, synthetic construct
NFSNDIMLLQLER

SEQ ID NO: 26 - protein, 11 aa, synthetic construct
HRRRKRSLDTS

SEQ ID NO: 27 - protein, 267 aa, synthetic construct
EDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGSLRENIELGNGPLEEAISALYYYSTGGTQLPTLARSFIVCIQMISEAARFQYIEGEMRTRIRYNRRSAPDPSVITLENSWGRLSTAIQESNQGAFASPIQLQRRNGSKFSVYDVSILIPIIALMVYRCAPPP

SEQ ID NO: 28 - protein, 697 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDVSFRLSGADPRSYGMFIKDLRNALPFREKVYNIPLLLPSVSGAGRYLLMHLFNYDGKTITVAVDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVTSKVVTSNIQLLLNTRNI

SEQ ID NO: 29 - protein, 703 aa, synthetic construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHRRRKRSLDTSDVSFRLSGADPRSYGMFIKDLRNALPFREKVYNIPLLLPSVSGAGRYLLMHLFNYDGKTITVAVDVTNVYIMGYLADTTSYFFNEPAAELASQYVFRDARRKITLPYSGNYERLQIAAGKPREKIPIGLPALDSAISTLLHYDSTAAAGALLVLIQTTAEAARFKYIEQQIQERAYRDEVPSLATISLENSWSGLSKQIQLAQGNNGIFRTPIVLVDNKGNRVQITNVTSKVVTSNIQLLLNTRNI

SEQ ID NO: 30 - protein, 218 aa, synthetic construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 709 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHRRRKRSLDTSDVSFRLSGATSSSYGVFISNLRKALPNERKLYDIPLLRSSLPGSQRYALIHLTNYADETISVAIDVTNVYIMGYRAGDTSYFFNEASATEAAKYVFKDAMRKVTLPYSGNYERLQTAAGKIRENIPLGLPALDSAITTLFYYNANSAASALMVLIQSTSEAARYKFIEQQIGKRVDKTFLPSLAIISLENSWSALSKQIQIASTNNGQFESPVVLINAQNQRVTITNVDAGVVTSNIALLLNRNNMA

SEQ ID NO: 32 - protein, 703 aa, synthetic construct; identical to SEQ ID NO: 29

SEQ ID NO: 33 - protein, 218 aa, synthetic construct; identical to SEQ ID NO: 30

SEQ ID NO: 34 - protein, 10 aa, synthetic construct
GYTFSSYWIE

SEQ ID NO: 35 - protein, 18 aa, synthetic construct
GEILPGGGDTNYNEIFKG

SEQ ID NO: 36 - protein, 10 aa, synthetic construct
TRRVPIRLDY

SEQ ID NO: 37 - protein, 15 aa, synthetic construct
KASQSVDYEGDSFLN

SEQ ID NO: 38 - protein, 7 aa, synthetic construct
AASNLES

SEQ ID NO: 39 - protein, 10 aa, synthetic construct
CQQSNEDPLT

SEQ ID NO: 40 - protein, 5 aa, synthetic construct
GGGGS

SEQ ID NO: 41 - protein, 8 aa, synthetic construct
DDDDKGGS

SEQ ID NO: 42 - protein, 5 aa, synthetic construct
DDDDK

SEQ ID NO: 43 - protein, 113 aa, synthetic construct
DVVVTQTPLSLPVSFGDQVSISCRSSQSLANSYGNTFLSWYLHKPGQSPQLLIYGISNRFSGVPDRFTGSGSGTDFTLKISTIKPEDLGMYYCLQGTHQPYTFGGGTKLEIKR

SEQ ID NO: 44 - protein, 121 aa, synthetic construct
EVQLQQSGTVLARPGASVKMSCKASGYRFTNYWIHWVKQRPGQGLEWIGGINPGNNYTTYKRNLKGKATLTAVTSASTAYMDLSSLTSEDSAVYYCTREGYGNYGAWFAYWGQGTLVTVSS

(19) United States
(12) Patent Application Publication
(10) Pub. No.: US 2025/0387478 A1
(43) Pub. Date: Dec. 25, 2025

(54) RECOMBINANT IMMUNOTOXIN COMPRISING A RIBOSOME INACTIVATING PROTEIN

(71) Applicant: ATB THERAPEUTICS, AYE (BE)

(72) Inventors: Max HOURY, Aye (BE); Bertrand MAGY, Aye (BE); Sébastien MERCX, Aye (BE); Sergej KIPRIJANOV, Aye (BE)

(21) Appl. No.: 18/839,507

(22) PCT Filed: Feb. 17, 2023
(86) PCT No.: PCT/EP2023/054107

§ 371 Date: Aug. 19, 2024

(30) Foreign Application Priority Data
Feb. 17, 2022 (EP) 22157321.5

(51) Int. Cl. A61K 39/395; A61K 38/16; A61K 39/00; A61P 35/00; C07K 14/415; C07K 16/28

(52) U.S. Cl. CPC A61K 39/39558; A61K 38/168; A61P 35/00; C07K 14/415; C07K 16/2803; A61K 2039/505; C07K 2317/24; C07K 2317/622; C07K 2319/00

(57) ABSTRACT

The present disclosure relates to a binder-toxin fusion protein comprising at least a protein binder, and a Ribosome-inactivating protein (RIP) type 1 or an active fragment thereof.

### CROSS-REFERENCE TO RELATED APPLICATIONS

This application is a U.S. national stage filing of International Application No. PCT/EP2023/054107, filed Feb. 17, 2023; which claims priority to EP Application Serial No. 22157321.5, filed Feb. 17, 2022. The entire contents of the above-referenced applications are hereby incorporated by reference.

### REFERENCE TO A SEQUENCE LISTING SUBMITTED ELECTRONICALLY

This application includes a Sequence Listing submitted electronically (Name: 5648_0030001_SequenceListing.xml; Size: 47,642 bytes; Creation Date: Apr. 7, 2025), which is hereby incorporated by reference in its entirety.

### FIELD OF THE DISCLOSURE

The present application relates to the field of a binder-toxin fusion proteins.

### BACKGROUND

Conjugates combining a target binder and a toxin have been developed forty years ago and now represent a major hope to fight cancer. These conjugates are mainly represented by the class of Antibody-Drug-Conjugates (ADC), consisting of a monoclonal antibody chemically conjugated to a chemical cytotoxic agent via a linker. These drugs combine the specificity of monoclonal antibodies to target cancer cells with the high toxic potency of the payload, to kill targeted cells, while sparing healthy tissues.

### SUMMARY

There is still a need for new such entities to provide better treatment options for different tumor types. It is hence one object of the present invention to provide such new entities.

It is one further object of the present invention to provide alternative or even better treatment options for cancer patients.

The methodologies used in the conception and reduction to practice of this invention are disclosed in PCT application PCT/EP2020/054263, the content of which is incorporated herein by reference in its entirety. The definitions and embodiments disclosed therein form part of the present disclosure. For clarity, the text of PCT application PCT/EP2020/054263 is appended to this application and forms part of its disclosure.

### BRIEF DESCRIPTION OF THE FIGURES

FIG. 1: A selection of possible binder-toxin fusion protein formats. All formats shown comprise an antibody Fc domain.

FIG. 2A: Alignment of different RIPs type I (Ribosome Inactivating Proteins type I). Some highly conserved residues (Tyr21, Phe24, Arg29, Tyr80, Tyr123, Gly140, Ala165, Glu177, Ala178, Arg180, Glu208, Asn209 and Trp211) are shown in bold.

FIGS. 2B and C: Further alignments and homology matrix of different RIPs type I

FIG. 3: Cell killing-activity of binder toxin protein comprising either a RIP I bryodin or alpha domain of ricin (RIP II) on target positive cell line. The antibody is anti-CD79B.

FIG. 4: Cell killing-activity of binder toxin protein comprising RIP I tricosanthin on target positive and target negative cell line. The antibody is anti-CD79B.

FIG. 5: Cell killing-activity of binder toxin protein comprising RIP I momordin on target positive cell line

FIG. 6: Evaluation of the expression of IgG-G4S-RIP type I toxins in transient expression in Nicotinia benthamiana All Binder toxin fusion proteins are well expressed (>200 mg/Kg fresh leave (this is oftentimes considered the threshold for industrialization).

FIG. 7: Cell killing-activity of binder-toxin fusion proteins comprising different RIP Type I on target positive cell line. The antibody is anti-CD79B (polatuzumab).

FIG. 8. Cell killing activity of binder-toxin fusion proteins comprising wildtype RIP I bryodin vs aglycosylated RIP I bryodin on a target positive cell line. The antibody is anti-CD79B (polatuzumab). It can be seen that deglycosylation of the toxin does not affect potency.

FIG. 9: Cell killing activity of binder-toxin fusion proteins comprising RIP I bryodin with a G4S linker (ATB 678) vs the same construct with the Liopt linker (ATB 679) on a target positive cell line (mammalian). ATB 673 is a naked antibody. The antibody is anti-CD22 inotuzumab. Liopt is a furin cleavable linker that is cleaved by mammalian cells while G4S is non-cleavable. Linker cleavability does yet not affect potency of the binder toxin fusion protein in this cell assay.

FIG. 10: Cell killing activity of binder-toxin fusion proteins comprising wildtype RIP I bryodin with the liopt linker on a target negative cell line. The antibody is anti-CD22 inotuzumab.

FIG. 11: Cell killing activity of binder-toxin fusion proteins comprising RIP I Momordin with a G4S linker on target positive cancer cells. The antibody binds to an antigen present on the surface of cancer cells.

FIG. 12: Protein synthesis inhibition in HELA cell lysate. Target independent activity of complete binder-toxin fusion proteins. In a HELA cell lysate, all the tested binder-toxin fusion proteins have similar activity. Deglycosylation of MOM and BD1 by mutating the glycosylation site does not impact activity.

FIG. 13: Internalization of constructs comprising the antibody anti-CD79B (polatuzumab) into CD79 positive cells. The binder toxin fusion protein comprising BD1 (ATB 639) is much better internalized than the naked antibody (ATB 372).

FIG. 14: Differences in pharmacokinetics between binder toxin fusion proteins comprising glycosylated toxin (ATB 639, ATB 657) vs binder toxin fusion proteins comprising deglycosylated toxin (ATB 752, ATB 662). While, as shown in FIGS. 8 and 12, deglycosylation does not affect activity negatively, it increases serum half life.

FIG. 15: Embodiments of the binder toxin fusion proteins according to the invention. Each combination of features between toxin, binder, linker and fusion site shall be deemed disclosed as an individual embodiment for which no list selections are to be made.

### DETAILED DESCRIPTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be construed as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

The embodiments of the present invention are shown in the claims.

According to a first aspect of the invention, a binder-toxin fusion protein is provided comprising at least
a) a protein binder, and
b) a ribosome-inactivating protein (RIP) type 1 or an active fragment thereof.

Ribosome-inactivating proteins (RIPs) are toxic N-glycosidases that depurinate eukaryotic and prokaryotic rRNAs, thereby arresting protein synthesis during translation. RIPs are widely present in various plant species and within different tissues. Those protein are known to play a key role in defense against pathogens and have been suggested to confer disease resistance. Plant based RIPs have so far been found in more than 50 different species from 14 families, including the Cucurbitaceae, Euphorbiaceae, Poaceae and Caryophyllales. In addition to plant, RIP have also been found in bacteria's, fungi, algae and even in mosquitoes.

RIPs constitute a large family of proteins that can be classified following their structural composition, RIP type I and type II.

Type I RIPs share a low molecular weight around 30 KD, resulting as single-chain proteins. The single-chain of type I RIPs consists of an enzymatically active domain (A domain or alpha domain) exerting N-glycosidase activity.

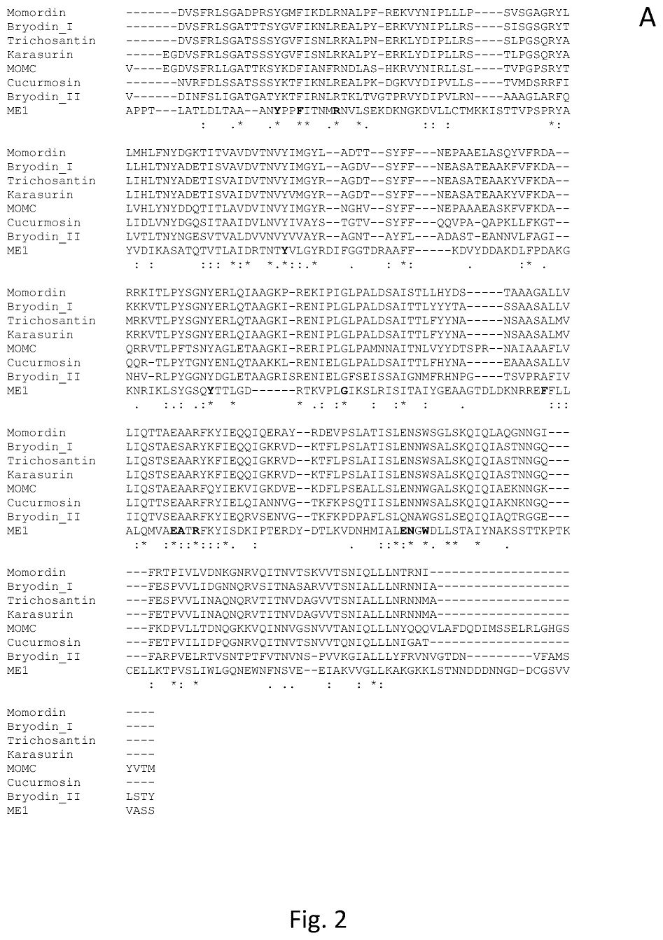

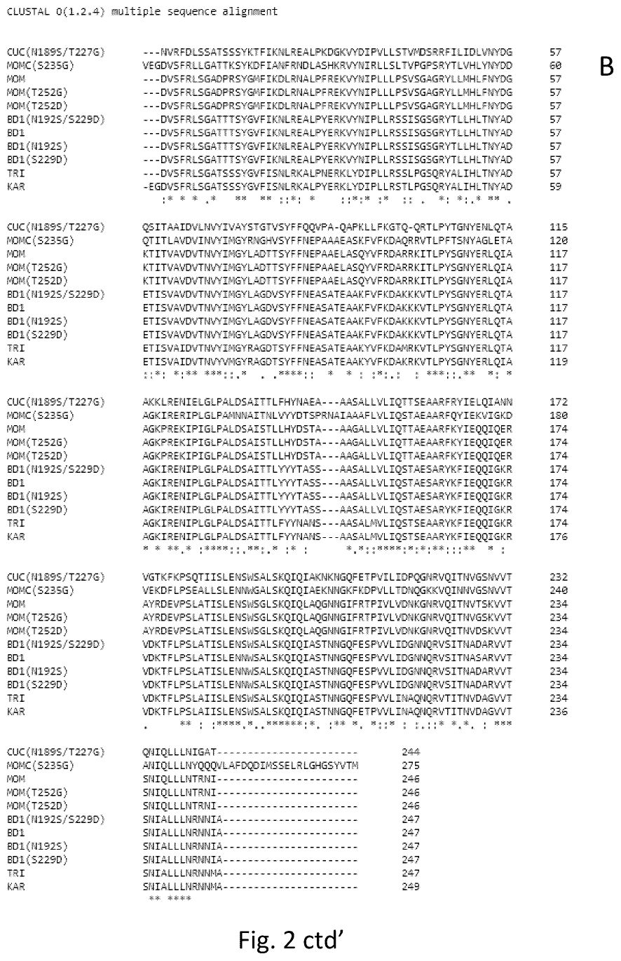

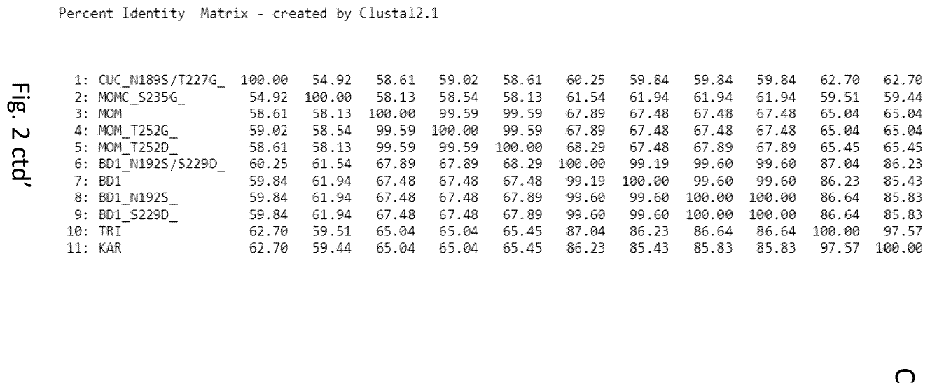

The amino acid sequences of Type I RIPs have particular sequence characteristics. In addition to several highly conserved hydrophobic amino acids, inter alia eleven_absolutely conserved residues exists in almost all Type I RIPs: Tyr21, Phe24, Arg29, Tyr80, Tyr123, Gly140, Ala 165, Glu177, Ala178, Arg180, Glu 208, Asn209 and Trp211. These residues are marked in the alignment in FIG. 2

Vivanco et al (1999) have shown that the plant type 1 RIP and ME1 and ME2 exhibit antibacterial activity against Agrobacterium tumefaciens and Agrobacterium radiobacter.

It is hence surprising that the inventors were able to produce RIP I based binder toxin fusion proteins in plant cells and plant hosts which were transfected with agrobacterium, irrespective of the alleged antibacterial activity of RIP I toxins.

Krishnan et al. (2002) demonstrated that RIP type I trichosanthin expressed in a transgenic tobacco plant conferred acquired resistance to pathogens as e.g. tobacco mosaic virus. The authors considered three possible explanations were considered for the degree of phenotypic abnormalities and low expression levels of RIPs in transgenic plants, namely (1) the mode of expression (constitutive vs. tissue-specific); (2) the targeting sequences (or lack thereof) within the coding genes; and (3) the variant toxicities of different RIPs and their effects on host plant ribosomes. RIP proteins vary greatly in their enzymatic and rRNA N-glycosidase activities, not to mention their activities against prokaryotic and/or eukaryotic ribosomes.

Krishnan et al. report that, while researchers have had a great deal of difficulty expressing most type I RIPs in transgenic plants, they have achieved high levels of expression of cereal seed ribosome inactivating proteins (RIPs), in transgenic tobacco and rice cultures. Cereal seed RIPs are intracellular proteins, which are not post-translationally cleaved, and exhibit no depurination activity against plant ribosomes in vitro or in vivo.

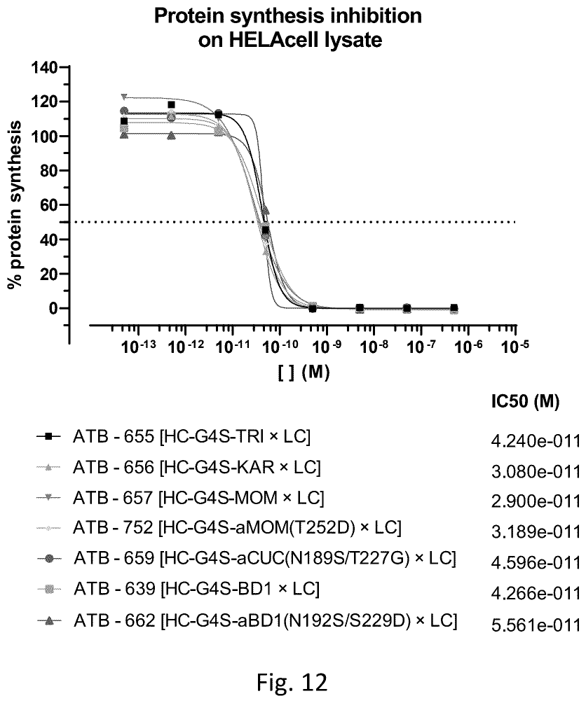

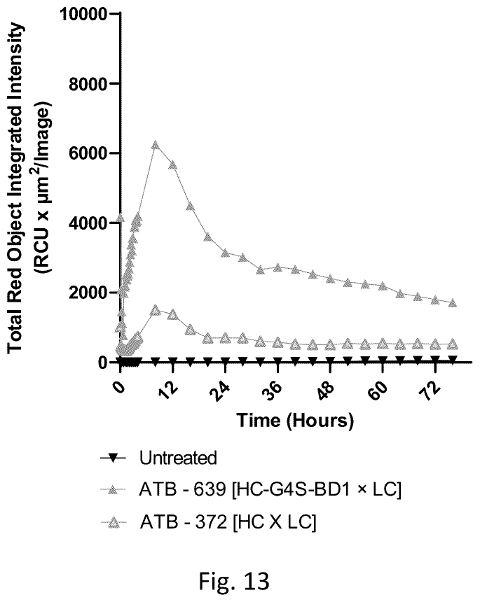

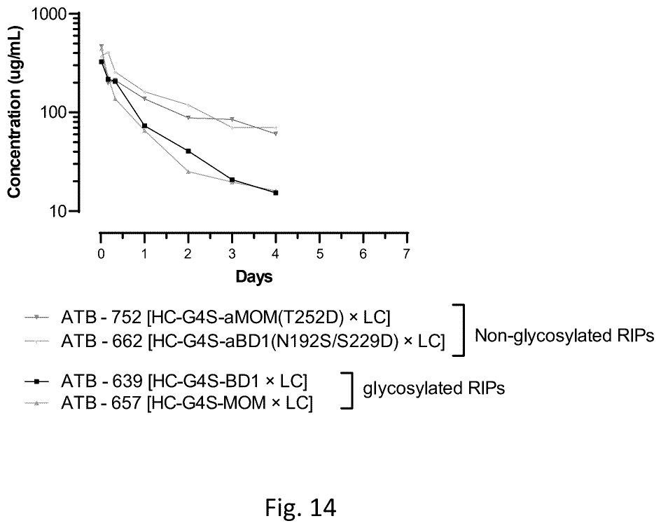

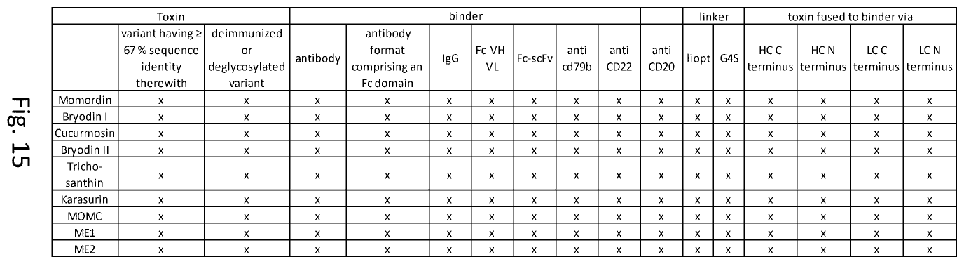

It is hence surprising the that the inventors found that, when bound to a protein binder, Type I RIPs can indeed be produced in transgenic plants even though the fusion construct as a whole retains ribosome inactivation activity, as the inventors have shown FIG. 12, where a binder toxin fusion protein IgG with Bryodin fused to the heavy chain still has ribosome inactivation activity on a lysate of antibody target negative Hela cells.

Type II RIPs are larger proteins with a weight comprised between 50-65 kDa, characterized by an enzymatically active A-chain and a slightly larger B chain (or beta chain, a lectin subunit) with galactose-like sugars.

In addition to RIPs type I and II, a third class—type III—is reported with few members bearing N-terminal domain which is correlative to the A domain of RIPs and fused to an unknown functional C-terminal domain. The C-terminal domains seems to be a protective feature to prevent self-inactivation. There are only few members, only found from barley and maize.

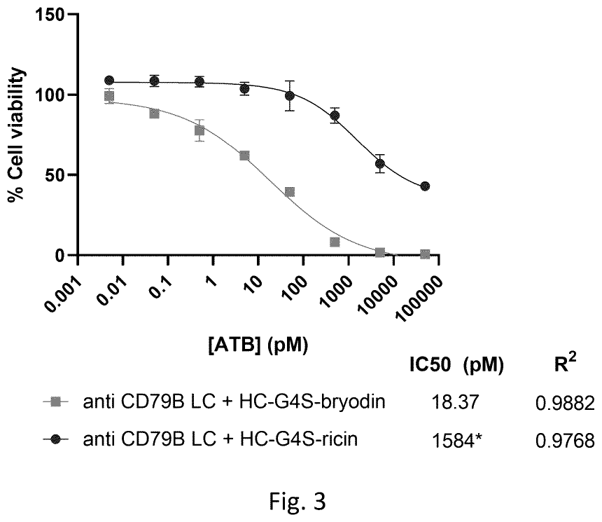

Interestingly, type I RIPs are less cytotoxic than their type II counterparts. The reduced cytotoxicity of type I RIPs is due to the absence of the cell-binding B chain (beta chain). It is hence surprising that the inventors found out that binder toxin fusion proteins comprising a type I RIP are extremely potent (see, inter alia, FIG. 3)

According to several embodiments, the Ribosome-inactivating protein (RIP) type 1 within the binder toxin fusion protein is at least one selected from the group consisting of:
Momordin (MOM)
Bryodin I (BD1)
Cucurmosin (CUC)
Bryodin II (BD2)
Trichosanthin (TRI)
Karasurin (KAR)
MOMC
ME1, and/or
ME2

In the following, more information is provided for these Ribosome-inactivating proteins, and examples are provided.

TABLE 1
The different RIP I toxins in the binder toxin
fusion protein according to the invention

| Ribosome- | | | SEQ ID NO |
|---|---|---|---|
| inactivating | | Uniprot identifier | herein |
| protein | Type | (example) | (example) |
| Momordin | Ribosome-inactivating | P16094 | 1 |
| | protein momordin I | | |
| Bryodin I | Ribosome-inactivating | P33185 | 2 |
| | protein bryodin I | | |
| Cucurmosin | Ribosome-inactivating | P84531 | 3 |
| | protein cucurmosin | | |
| Bryodin II | Ribosome- | P98184 | 4 |
| | inactivating protein | | |
| | bryodin II | | |
| Trichosanthin | Ribosome- | P09989 | 5 |
| | inactivating protein | | |
| | alpha-trichosanthin | | |
| Karasurin | Ribosome- | P24478 | 6 |
| | inactivating protein | | |
| | karasurin-C | | |
| MOMC | n/a | A0A6J1DRD7 | 7 |
| ME1 | n/a | n/a | 8 |
| ME2 | n/a | n/a | n/a |

The sequences of ME1 and ME2, as well as the characteristics thereof, are disclosed in Vivanco et al (1999).

According to several embodiments, the Ribosome-inactivating protein (RIP) comprises at least one amino acid sequence selected from the group consisting of SEQ ID NO 1-8, or a homologue thereof having at least 66% sequence identity therewith.

In several embodiments, the toxin sequence has a sequence identity of ≥67%; ≥68%; ≥69%; ≥70%; ≥71%; ≥72%; ≥73%; ≥74%; ≥75%; ≥76%; ≥77%; ≥78%; ≥79%; ≥80%; ≥ 81%; ≥82%; ≥83%; ≥84%; ≥85%; ≥86%; ≥87%; ≥88%; ≥89%; ≥90%; ≥91%; ≥92%; ≥93%; ≥94%; ≥95%; ≥96%; ≥97%; ≥98%; ≥99%, and most preferably 100% with any one of SEQ ID NO 1-8.

In particular, mutated variants of these toxins which retain toxic functionality are likewise encompassed. Such mutated variants can for example comprise mutations/substitutions in N-glycosylation motifs (e.g. N-X-S or N-X-T, in which X can't be P) to produce deglycosylated variants of the toxin, or can comprise mutations/substitutions to deimmunize the respective toxin (see e.g. Zinsli et al 2020, the content of which is incorporated herein by reference for enablement purposes).

Deglycosylated variants of BD1 can for example comprise one or more of the following substitutions: N192S, A228V, S229D, S229G, R230G, A230S, and/or R231D. Deglycosylated variants of MOM can for example comprise one or more of the following substitutions: T252G, T252D, S253A, K254G, K254D, D1A, and/or D1S. Deglycosylated variants of CUC can for example comprise one or more of the following substitutions: N189S, T227G.

The inventors have surprisingly shown that deglycosylated variants may have improved pharmacokinetics, as well as prolonged serum half-life. Without being bound to theory, this may be due to reduced hepatic clearance caused by the lacking glycosyls.

Interestingly, while quite a few of the currently approved protein pharmaceuticals need to be properly glycosylated to exhibit optimum therapeutic efficacy (because glycosylation can influence a variety of physiological processes at both the cellular and protein levels (e.g. protein-protein binding, protein molecular stability), such effects do not seem to play a role in the toxin parts of the binder toxin proteins according to the invention. The inventors have realized this fact, and hence established a feasible pathway to improve pharmacokinetics, and serum half-life, without compromising efficacy.

According to one embodiment, the Ribosome-inactivating protein (RIP) type 1 within the binder toxin fusion protein is Momordin, or a variant thereof being deimmunized, deglycosylated or having a sequence identity to Momordin as specified above.

According to one embodiment, the Ribosome-inactivating protein (RIP) type 1 within the binder toxin fusion protein is Bryodin 1, or a variant thereof being deimmunized, deglycosylated or having a sequence identity to Bryodin as specified above.

According to one embodiment, the protein binder is selected from the group consisting of
an antibody
an antibody fragment or derivative retaining target binding capacity, or
an antibody mimetic.

According to one embodiment, the binder-toxin fusion protein comprises a peptide linker connecting the binder, or a domain thereof, with the toxin, or with a cleavable domain comprised in the toxin.

According to several embodiments of the binder-toxin fusion protein
the peptide linker or the cleavable domain is specifically or non-specifically cleavable by an enzyme expressed by a mammalian cell, or an enzyme that is produced by a mammalian host,
the peptide linker or the cleavable domain is not cleavable by an enzyme expressed by a plant cell, or an enzyme that is produced by a plant host, and/or
the binder-toxin fusion protein is expressed in a transfected plant cell or transfected plant host.

The skilled person has a bunch of routine methods at hand to check whether the condition that peptide linker or the cleavable domain in the toxin is not cleavable by an enzyme expressed by a plant cell, or an enzyme that is produced by a plant host, is met. See e.g., Wilbers et al (2016). Also, the skilled person can check with routine methods whether the peptide linker or the cleavable domain is specifically or non-specifically cleavable by an enzyme expressed by a mammalian cell, or an enzyme that is produced by a mammalian host,

The transfection of the plant cell or plant host can be transient or stable.

According to one embodiment, the binder-toxin fusion protein comprises a non-cleavable peptide linker connecting the binder, or a domain thereof, with the toxin.

According to one embodiment, the protein binder binds to human CD20, human CD22 or human CD79B.

CD79b (B-cell antigen receptor complex-associated protein β-chain) is a surface protein and involved in the humoral immune response.

CD79b is produced by B cells. It binds to CD79a and is linked to it by disulfide bridges Two of these heterodimers bind to membrane-bound antibodies of subtypes mIgM or mIgD to form the B cell receptor (BCR) to which antigens bind. CD79b enhances the phosphorylation of CD79a. Following antigen binding, the antigen-antibody BCR is endocytosed. CD79b is glycosylated. It has an ITAM motif intracellularly that binds and is phosphorylated by the protein kinases Syk or Lyn following activation of the BCR

The full sequence of CD79b has for the first time been disclosed in 1994.

Protein binders to CD79B have been described in the art. The first antibody (murine) against CD79b is called SN8, and has been published by Okazaki et al (1993).

Polson et al (2007) have discussed the possibility to make Antibody drug conjugate (ADCs) or recombinant immunotoxins against CD79b.

The first humanized anti CD79b antibody (Polatuzumab) is disclosed in U.S. Pat. No. 8,545,850. In this patent, an ADC consisting of MMAE linked to Polatuzumab is also disclosed.

B-lymphocyte antigen CD20 or CD20 is expressed on the surface of all B-cells beginning at the pro-B phase (CD45R+, CD117+) and progressively increasing in concentration until maturity. In humans CD20 is encoded by the MS4A1 gene. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes a B-lymphocyte surface molecule that plays a role in the development and differentiation of B-cells into plasma cells. This family member is localized to 11q12, among a cluster of family members. Alternative splicing of this gene results in two transcript variants that encode the same protein. The protein has no known natural ligand and its function is to enable optimal B-cell immune response, specifically against T-independent antigens. It is suspected that it acts as a calcium channel in the cell membrane. CD20 is induced in the context of microenvironmental interactions by CXCR4/SDF1 (CXCL12) chemokine signaling and the molecular function of CD20 has been linked to the signaling propensity of B-cell receptor (BCR) in this context.

CD20 was discovered by Lee Nadler from the Dana Farber Cancer Institute in 1980.

Protein binders to CD20 have been described in the art. The last three decades have seen considerable progress in understanding of the structure and function of the CD20 molecule and in the development of engineered anti-CD20 mAb. Information on anti-CD20 antibodies can for example be found in Lim et al (2010), the content of which is incorporated herein by reference for enablement purposes. The following table shows some anti CD20 antibodies known to the skilled artisan:

TABLE 2
anti CD20 antibodies that can be used in the fusion
protein according to an embodiment of the invention

| | | | First | |
|---|---|---|---|---|
| INN | alias | Format | approval | Reference |
| Rituximab | C2B8 | Chimeric IgG1 | 1997 | Reff et al 1994 |
| Ofatumumab | | Human IgG1 | 2009 | Hagenbeek et |
| | | | | al 2008 |
| Ocrelizumab | | Humanized | 2017 | Genovese et al |
| | | IgG1 | | 2008 |
| Veltuzumab | | Humanized | 2015 | Milano & |
| | | IgG1 | | Castillo 2009 |
| Obinutuzumab | GA101 | Humanized | 2013 | Umana et al |
| | | IgG1 | | 2006 |

The content of the references mentioned is incorporated herein by reference for enablement purposes.

CD22, or cluster of differentiation-22, is a molecule belonging to the SIGLEC family of lectins. It is found on the surface of mature B cells and to a lesser extent on some immature B cells. CD22 is a transmembrane protein with a molecular weight of 140 kDa. The extracellular part of CD22 consists of seven immunoglobulin domains and the intracellular part is formed by 141-amino acid cytoplasmic tail.

Because CD22 is restricted to B cells, it has been considered as a target for immunotherapy of B cell malignancies. There are several mechanisms by which this can be achieved, namely monoclonal antibodies, bispecific antibodies, antibody-drug conjugates, radioimmunoconjugates or CAR-T cells.

The following table shows some anti CD22 antibodies known to the skilled artisan:

TABLE 3
anti CD22 antibodies that can be used in the fusion
protein according to an embodiment of the invention

| | | | First | |
|---|---|---|---|---|
| INN | | Format | approval | Reference |
| Moxetumomab | CAT-8015 | | 2018 | Onda et al 2011 |
| Inotuzumab | | Humanized | 2017 | Wynne et al |
| | | IgG4 | | 2019 |

The content of the references mentioned is incorporated herein by reference for enablement purposes.

According to one aspect, such binder-toxin fusion protein is one of the formats selected from the group consisting of
(scFv-Fc)-(linker)-toxin (dimer)
toxin-(linker)-Fc-VH/VL
tetramer of two HC and two LC-(linker)-toxin (IgG format)
tetramer of two LC and two HC-(linker)-toxin (IgG format), or
tetramer of two LC-(linker)-toxin and two HC-(linker)-toxin (IgG format)
   wherein the linker is optional.

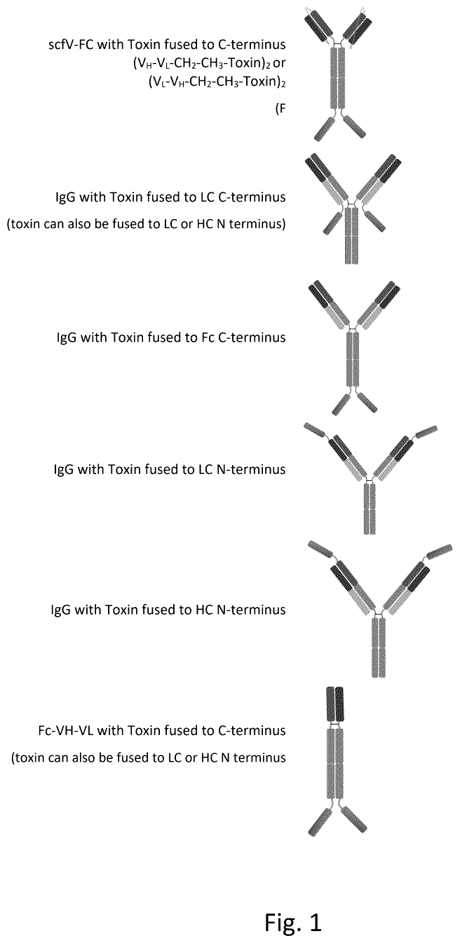

FIG. 1 shows a selection of embodiments of binder-toxin fusion protein formats according to an embodiment of the invention
CH₃=heavy chain constant domain 3
CH₂=heavy chain constant domain 2
V_{L}=light chain variable domain
V_{H}=heavy chain variable domain
Fc=antibody Fc domain
LC=light chain
HC=heavy chain

As can be seen, all embodiments shown in FIG. 1 comprise an Fc domain. Such domain is for example lacking in antibody fragments like scFv, Fab or (Fab) 2. Hence, in one embodiment of the binder toxin fusion protein, the antibody or fragment therein comprises at least one Fc domain

In addition to what is shown in FIG. 1, toxin molecules can also be fused to the heavy chain and the light chains simultaneously.

Also, the protein binder can be bispecific. In one such embodiment, the antibody can be designed in such way that one VF/VL pair binds to target 1 and the other one binds to target 2 Such approaches are called “quadroma”, or further developed as “knobs into holes”. In another such embodiment, two different VH domains can be chimerized, as well as two different VL domains, so as to create a dual variable domain antibody (called “DVD-Ig”). See for example Brinkmann and Kontermann (2017), the content of which is incorporated herein by reference for enablement purposes.

Further the binder toxin fusion can comprise two different toxins fused, e.g. to the two chains of the antibody.

In another embodiment where the protein binder comprises two or more chains it may be provided that two nucleic acid constructs are provided, the first comprising the three polynucleotides encoding for the first chain of the protein binder, the linker and the toxin, while the second comprises the polynucleotide encoding for the second chain of the protein binder.

Both transient and stable expression could be induced by an “inducible promoter”. These promoters selectively express an operably linked DNA sequence following to the presence of an endogenous or exogenous stimulus or in response to chemical, environmental, hormonal, and/or developmental signals. These regulatory elements are, without limitation, sensitive to ethanol, heat, light, stress, jasmone, salicylic acid, phytohormones, salt, flooding or drought, as reviewed by Abdel-Ghany et al (2015) and discussed in U.S. Pat. No. 10,344,290 B2, both of which are incorporated herein by reference. Inducible promotors including, but not limited to, synthetic components discuss in Ali et al (2019), the content of which is incorporated herein by reference.

According to an embodiment of the invention, the plant or plant cell is from the genus Nicotiana.

The genus Nicotiana encompasses tobacco plants. Tobacco plants or plant cells have already been tested to produce recombinant immunotherapeutic binder-toxin fusion proteins composed of a small sFv fragment linked to a protein toxin with a stable linker (Francisco et al. (1997), and U.S. Pat. No. 6,140,075A.

According to one further embodiment of the invention, the plant cell is at least one selected from the group consisting of:
Nicotiana tabacum cv. BY2,
Nicotiana tabacum NT-1,
Arabidopsis thaliana,
Daucus carota, and/or
Oryza sativa.

Nicotiana tabacum cv. BY2 aka Tobacco BY-2 cells and cv. Nicotiana tabacum 1 (NT-1, a sibling of BY-2) are nongreen, fast growing plant cells which can multiply their numbers up to 100-fold within one week in adequate culture medium and good culture conditions. This cultivar of tobacco is kept as a cell culture and more specifically as cell suspension culture (a specialized population of cells growing in liquid medium, they are raised by scientists in order to study a specific biological property of a plant cell). In cell suspension cultures, each of the cells is floating independently or at most only in short chains in a culture medium. Each of the cells has similar properties to the others.

The model plant system is comparable to Hela cells for human research. Because the organism is relatively simple and predictable it makes the study of biological processes easier and can be an intermediate step towards understanding more complex organisms. They are used by plant physiologists and molecular biologists as a model organism, and also used as model systems for higher plants because of their relatively high homogeneity and high growth rate, featuring still general behavior of plant cell. The diversity of cell types within any part of a naturally grown plant (in vivo) makes it very difficult to investigate and understand some general biochemical phenomena of living plant cells. The transport of a solute in or out of the cell, for example, is difficult to study because the specialized cells in a multicellular organism behave differently. Cell suspension cultures such as tobacco BY-2 provide good model systems for these studies at the level of a single cell and its compartments because tobacco BY-2 cells behave very similarly to one another. The influence of neighboring cells behavior is in the suspension is not as important as it would be in an intact plant. As a result, any changes observed after a stimulus is applied can be statistically correlated and it could be decided if these changes are reactions to the stimulus or just merely coincidental. BY-2 and NT-1 cells are relatively well understood and often used in research, including the expression of heterologous proteins, in particular antibodies (Hellwig et al (2004). Such methods are disclosed in Häkkinen et al. (2018), the content of which is incorporated herein by reference.

Torres (1989) discusses methods to establish Carrot Cell Suspension Cultures (Daucus carota). Shaaltiel et al (2007) discuss the production of enzymes using a carrot cell based expression system. The content of these articles is incorporated herein by reference. Daucus carota and Oryza sativa are also discussed as suitable plant-cell based expressions systems in Santos et al (2016), the content of which is incorporated herein by reference. The Production of recombinant proteins in Nicotiana tabacum, Arabidopsis thaliana, Oryza sativa is disclosed in Plasson et al (2009), the content of which is incorporated herein by reference.

Generally, the present invention can be practiced with any plant variety for which cells of the plant can be transformed with an DNA construct suitable for expression of a foreign polypeptide and cultured under standard plant cell culture conditions. Plant cells suspension or plant tissues culture is preferred, although callus culture or other conventional plant cell culture methods may be used.

According to one other embodiment of the invention, the plant is Nicotiana benthamiana. The production of antibodies in Nicotiana plants is for example disclosed in Daniell et al. (2001), the content of which is incorporated herein by reference.

Other plants or plant cells that can be used in the context of the present invention include, but are not limited to, lettuce (Lactuca spp.), spinach (Spinacia oleracea), and Arabidopsis (Arabidopsis spp).

In several embodiments, the cleavage site is selected from the group consisting of
a) Endosomal and/or Lysosomal proteases cleavage site
b) Cytosolic protease cleavage site, and/or
c) Cell surface proteases cleavage site.

Examples of such enzymes and their cleavage sites are shown in the following table (see also Choi et al (2012), the content of which in incorporated by reference herein. Reference is made, in this table, to the “Merops” database for more enabling information as regards the respective enzymes. https://www.ebi.ac.uk/merops/index.shtml.

TABLE 4
Enzymes and their cleavage sites

| | | | cleavage sequence | |
|---|---|---|---|---|
| | | | (one letter code) | |
| | | | general motif | |
| | | | (examples only) X | |
| | | | can be any naturally | |
| | | | proteinogenic | |
| Class | Enzyme class | example | amino acid | reference |
| “Linker | Proprotein | Furin | RXR/KR↓S/A/G/Nxxx | merops S08.071 |
| class 1” | convertase | | | |
| Endosomal | subtilisin/kexin | | | |
| and/or | family | | | |
| Lysosomal | Cathepsins | Cathepsin B | xxF/xV/R/G/L/S/A↓ | merops C01.060 |
| Cleavage | | | A/F/Lxxx | |
| site | | Cathepsin E | xxxL/F↓Vxxx | merops A01.010 |
| | | Cathepsin D | xxxL/F↓xxxx | merops A01.009 |
| | | Cathepsin L | xxL/V/F/IR/K↓S/A/Gxxx | merops C01.032 |
| | | Cathepsin K | xK/R/GF/L/I/V/Px↓xxxx | merops C01.036 |
| | | Cathepsin C | xSxE/S↓xxxG/R | merops C01.070 |
| “Linker | Caspases | Caspase 3 | DxxD↓A/G/S/Txxx Or | merops C14.003 |
| class 2” | | | xxxx↓GGFV | |
| Cytosolic | | Caspase 8 | D/LxxD↓G/S/Axxx | merops C14.009 |
| cleavage | Kallikereins | hK 1 | xxF/IR/Y↓R/SxGx | merops S01.160 |
| site | (hK) | hK 2 | G/K/AxxR↓xxxG/S/T | merops S01.161 |
| | | hK3 | S/IS/QxY/Q/R↓SSxx | |
| | | hK10 | No determined | |
| “Linker | Matrix metallo | MMP2 | xP/Axx↓L/Ixxx | merops M10.003 |
| class 3” | proteases | MMP1 | XP/Axx↓L/Ixx | merops M10.001 |
| Cell | | MMP3 | xxxR/N/G↓L/Kxx | merops S01.072 |
| surface | | MMP7 | xPA/G/Lx↓Lxxx | merops M10.005 |
| cleavage | | MMP8 | GP/A/Sxx↓Lxxx | merops M10.002 |
| site | | MMP9 | GP/Axx↓Lxxx | merops M10.004 |
| | | | P2 is preferably a L | |
| | | | P1 is preferably a G | |
| | | MMP12 | GP/A/GL/A/Gx↓Lxxx | merops M10.009 |
| | | MMP14 | xPxx↓Lxxx | merops M10.014 |
| | Matriptase | Matriptase 2 | xxxR↓k/G/Rxxx | merops S01.308 |
| | | Matriptase 1 | xxxR↓K/V/A/RVxx | merops S01.302 |
| | tissue-type | Urokinase | xSG/SR/K↓xR/Vxx | merops S01.231 |
| | plasminogen | type | | |
| | activator | plasminogen | | |
| | | activator | | |
| | | (uPA) | | |

The cleavage site is described from the cleavage site point (represented by ↓). The letter x refers to all amino acids. Where there are several preferential amino acid, there are separated by a slash (/).

Such enzyme is preferably a protease. In one embodiment, said peptide linker is not cleavable by a plant enzyme.

Furin is an enzyme which belongs to the subtilisin-like proprotein convertase family, and cleaves proteins C-terminally of the canonic basic amino acid sequence motif Arg-X-Arg/Lys-Arg (RX(R/K) R), wherein X can be any naturally proteinogenic amino acid. Said motif is called a furin cleavage site herein. Preferably, the sequence thereof is HRRRKRSLDTS (also called “liopt” herein).

Cathepsins are proteases found in all animals as well as other organisms. Most of the members become activated at the low pH found in lysosomes. Cathepsin B is capable of cleaving a peptide sequence which comprises the dipeptide motif Val-Ala (VA). Said motif is called a Cathepsin B cleavage site herein. The skilled artisan finds sufficient enabling information on cathepsins and their cleavage sites in Turk et al (2012), the content of which is incorporated herein by reference.

Caspases (cysteine-aspartic proteases, cysteine aspartases or cysteine-dependent aspartate-directed proteases) are a family of protease enzymes playing essential roles in programmed cell death. Over 1500 caspase substrates have been discovered in the human proteome. The general cleavage motif is DXXD-A/G/S/T, wherein X can be any naturally proteinogenic amino acid. The skilled artisan finds sufficient enabling information on caspases and their cleavage sites in Kumar el al (2014), the content of which is incorporated herein by reference.

Matrix metalloproteinases (MMPs), also known as matrixins, are calcium-dependent zinc-containing endopeptidases; other family members are adamalysins, serralysins, and astacins. Collectively, these enzymes are capable of degrading all kinds of extracellular matrix proteins, but also can process a number of bioactive molecules. The skilled artisan finds sufficient enabling information on Matrix Metallo Proteases and their cleavage sites in Eckard et al (2016), the content of which is incorporated herein by reference.

Generally, the skilled artisan is capable, by routine considerations and literature referral, to select specific cleavage sites that match with the respective mammalian enzyme, to control target specific release of the protein toxin or protoxin. General guidelines to find these cleavage sites are e.g. disclosed in Rawlings (2016).

According to one embodiment of the invention, the protein toxin or protoxin is a de-immunized variant of a native protein toxin. Recombinant methods to de-immunize protein toxins by sequence modification are disclosed, e.g., in Schmohl et al. (2015), or Grinberg and Benhar (2017), the content of which is incorporated by reference herein.

In one embodiment, said protein toxin or protoxin is not toxic to plants or plant cells. The skilled person has a bunch of routine methods at hand to check whether this condition is met. See e.g., Klaine and Lewis (1995) for an overview, the content of which is incorporated by reference herein.

According to one embodiment of the invention, said protein comprises at least one plant-specific N-glycan. N-glycans are glycans that are linked to the amide group of asparagine (Asn) residues in a protein, mostly in an Asn-X-Thr or Asn-X-Ser (NXT or NXS) motif, where X is any amino acid except proline. Typical plant-specific N-glycans are disclosed in Gomord et al. (2010), and differ significantly from mammalian N-glycan patterns.

It is in this respect important to stress that N-Glycans produced by plants are markedly different from those produced, e.g., in mammals. In particular, N-Glycans produced by tobacco plants have
a Fucose residue conjugated to the proximal N-Acetyl-Glucosamine residue via a α3 glycosidic link (instead of α6 as in mammals)
a Xylose residue conjugated to the proximal Mannose residue via a β2 glycosidic link
two distal N-Acetyl-Glucosamine residues, each of which carry a Fucose residue via a α3 glycosidic link, and a Galactose residue via a β3 glycosidic link (instead of a neuraminic acid in mammals).

On the other hand, proteins recombinantly expressed in e.g. algae often lack any kind of glycosylation. Algae are however capable of expressing IgG shaped antibodies, or antibody fragments having a one or more disulfide bridges.

The major plant-based glycoforms identified are complex type glycans (GnGn/GnGnXF). Other glycoforms (Man5-Man9, GnGnF, GnGnX, MMXF, Man5Gn and GnM(X)(F)) can be detected as well.

According to this nomenclature, MGnX means for example

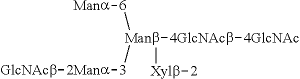

Background on methods for analyzing peptide glycoforms is provided in WO2020169620, the content of which is incorporated herein for enablement purposes.

According to another aspect of the invention, a pharmaceutical composition comprising at least the binder-toxin fusion protein according to the above description is provided, which optionally comprises one or more pharmaceutically acceptable excipients.

According to another aspect of the invention, a combination comprising (i) the binder-toxin fusion protein or the pharmaceutical composition according to the above description, and (ii) one or more further therapeutically active compounds, is provided.

According to another aspect of the invention, the binder-toxin fusion protein, the composition or the combination according to the above description is provided for (the manufacture of a medicament for) use in the treatment of a human or animal subject
suffering from,
being at risk of developing, and/or
being diagnosed for,
   developing a neoplastic disease, or for the prevention of such condition.

This language is deemed to encompass both the swiss type claim language accepted in some countries (in this case, brackets are deemed absent) and EPC2000 language (in this case, brackets and content within the brackets is deemed absent).

According to another aspect of the invention, a method for treating a human or animal subject
suffering from,
being at risk of developing, and/or
being diagnosed for
   developing a neoplastic disease, or for the prevention of such condition is provided, said method comprising the administration of a therapeutically effective amount of the binder-toxin fusion protein, the composition or the combination according to the above description

### Definitions

“Percentage of sequence identity” as used herein, is determined by comparing two optimally aligned biosequences (amino acid sequences or polynucleotide sequences) over a comparison window, wherein the portion of the corresponding sequence in the comparison window may comprise additions or deletions (i.e., gaps) as compared to the reference sequence, which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides that are substantially identical to the polypeptides exemplified herein. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

The terms “protein toxin” or “protein protoxin”, refer without limitation to toxins that are, by their chemical nature, proteins (i.e., peptides having a length of ≥50 amino acid residues) or polypeptides (i.e., peptides having a length of ≥10-≤50 amino acid residues). A protoxin, in the meaning of the present invention, is a precursor of a toxin, also called a latent toxin, which needs to be activated, e.g., by cleaving off an inhibitory amino acid sequence, or by undergoing a conformational change. The terms “protoxin” and “protein protoxin” are used interchangeably here and mean the same subject matter.

The term “fusion protein” as used herein refers to a protein that has a peptide component operably linked to at least one additional component and that differs from a natural protein in the composition and/or organization of its domains.

The term “operably linked” as used herein, when referring to two or more polynucleotides, means a situation when the different polynucleotides are placed in a functional relationship with one another. For instance, a promoter is operably linked to a coding sequence if the promoter effects the transcription of the coding sequence. Likewise, the coding sequence of a signal peptide is operably linked to the coding sequence of a polypeptide if the signal peptide effects the extracellular secretion of that polypeptide. According to one embodiment of the present invention, when the respective polynucleotides encode different peptides, “operably linked” means that the respective polynucleotides are contiguous and, where necessary to join two protein coding regions, the open reading frames are aligned.

The term “cleavable peptide linker” as used herein refers to an internal amino acid sequence within the fusion protein which contains residues linking the binder moiety and toxin protein so as to render the toxin protein incapable of exerting its toxic effect outside the target cell or limiting its ability of toxin protein to inhibit cell growth (cytostasis) or to cause cell death (cytotoxicity). In such way, the protein toxin is maintained inactive as long as it is in the plasma, until it reaches the target cell, where the cytotoxic payload will be selectively released and/or activated (Grawunder & Stein, 2017). Inside the target cell, the cleavable linker sequence is cleaved and the toxin protein becomes active or toxic. The fusion protein of the invention is composed of a cell-specific binder moiety and an protein toxin moiety linked by a a specific amino acid residue or amino acid sequence that has cleavage recognition site for specific proteases, particularly but not limited to cancer specific protease, and/or are cleavable under specifics conditions such as, without limitation, acid and/or reducing conditions. Sequences encoding cleavage recognition sites for specific protease may be identified among known ubiquitous human protease and/or by testing the expression of cancer associate protease. Also the linker sequence should not interfere with the role of the binder moiety in cell binding and internalization into lysosomes.

The term “cleavable domain” of a toxin relates to a sequence that, once cleaved by hydrolysis or enzymatic cleavage, activates the toxin part of the toxin. Many toxins have an amino acid domain that is specifically cleaved by an enzyme, or by pH dependent hydrolysis (e.g. after endocytosis in the endosomes), so as to release the active toxin part into the cytosol. Such cleavable domains double act as “naturally occurring” cleavable peptide linkers (or “intrinsic cleavage sites”), contrary to the cleavable peptide linkers which have to be used in case the toxin does not comprise a cleavable domain for activation.

Hence, while a cleavable linker may provide, under particular circumstances, advantages over a stable linker as regards the activity profile, the use thereof complicates the production of respective binding protein-toxin conjugates in mammalian, insect and yeast cells, because cleavage of the linker leads to toxin release from the protein binder, resulting of self-intoxication of the production system. This, however, does not apply to plant-based production systems, because
(i) they don't cleave the linker (due to lack of respective proteases or reducing/hydrolyzing conditions) and/or
(ii) the respective protein toxin which is toxic to mammals or mammalian cells is not toxic to plants or plant cells.

However, the inventors have experienced that even binder toxin fusion proteins with not cleavable by mammalian cells can pose a problem when the production thereof in mammalian cells is tested. Without being bound by theory, this may due to the fact that immediately after intracellular protein expression, the Ribosome Inactivating Protein component, although bound to the antibody, may exert toxic effects on the ribosomes of the expressor cells. Such negative effects were not encountered when a non-mammalian expression system was used, like e.g. in plant or plant cells (e.g. Nicotiana, as disclosed elsewhere herein), which, without being bound to theory, might be due to differences in ribosome structure between plant cells and mammalian cells.

It should be noted that cytosolic ribosomes in eukaryotes consist of a 60S large subunit (LSU) and a 40S small subunit (SSU). The latter decodes mRNA, and the former catalyzes the peptidyl transferase reaction that leads to the peptide bond formation of the newly synthesized proteins. The subunits are composed of rRNA and accessory ribosomal proteins (RPs). The large subunit is composed of 5S, 5.8S, and 25S rRNA, which ranges between 25S and 26S in plant cells, yet it is 28S in mammalian cells.

As used herein, the term “antibody” shall refer to an antibody composition having a homogenous antibody population, i.e., a homogeneous population consisting of a whole immunoglobulin, or a fragment or derivative thereof retaining target binding capacities.

Particularly preferred, such antibody is an IgG antibody, or a fragment or derivative thereof retaining target binding capacities. Immunoglobulin G (IgG) is a type of antibody. Representing approximately 75% of serum antibodies in humans, IgG is the most common type of antibody found in blood circulation. IgG molecules are created and released by plasma B cells. Each IgG has two antigen binding sites.

IgG antibodies are large molecules with a molecular weight of about 150 kDa made of four peptide chains. It contains two identical class y heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. The Fc regions of IgGs bear a highly conserved N-glycosylation site. The N-glycans attached to this site are predominantly core-fucosylated diantennary structures of the complex type. In addition, small amounts of these N-glycans also bear bisecting GlcNAc and α-2,6-linked sialic acid residues.

There are four IgG subclasses (IgG1, 2, 3, and 4) in humans, named in order of their abundance in serum (IgG1 being the most abundant).

As used herein, the term “antibody fragment” shall refer to fragments of such antibody retaining target binding capacities, and which, in one embodiment, still comprise an Fc domain, or a CH2 or CH3 domain only.

As used herein, the term “derivative” shall refer to protein constructs being structurally different from, but still having some structural relationship to the common antibody concept, e.g., scFv-Fc, Fc-VH/VL, and DVD-Ig, as well as bi-, tri- or higher specific antibody constructs or monovalent antibodies, and further retaining target binding capacities. All these items are explained below.

Other antibody derivatives known to the skilled person are IgAs (two IgG structures joined by a J chain and a secretory component), shark antibodies, antibodies consisting of new world primate framework plus non-new world primate CDR, dimerised constructs comprising CH3+VL+VH,

Methods for the production of a hybridoma cell have been previously described (see Köhler and Milstein 1975, incorporated herein by reference). Essentially, e.g., a mouse is immunized with a human soluble Guanylyl Cyclase (sGC) protein, followed by B-cell isolation from said mouse and fusion of the isolated B-cell with a myeloma cell.

Methods for the production and/or selection of chimeric or humanized mAbs are known in the art. Essentially, e.g., the protein sequences from the murine anti sGC antibody which are not involved in target binding are replaced by corresponding human sequences. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies. All of these disclosures are incorporated herein by reference.

Methods for the production and/or selection of fully human mAbs are known in the art. These can involve the use of a transgenic animal which is immunized with human sGC, or the use of a suitable display technique, like yeast display, phage display, B-cell display or ribosome display, where antibodies from a library are screened against human sGC in a stationary phase.

In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in EP1480515A2 by TaconicArtemis. All of these disclosures are incorporated herein by reference.

IgG, scFv-Fc, and Fc-VH/VL are antibody formats well known to the skilled person. Related enabling techniques are available from the respective textbooks.

As used herein, the term “scFv-Fc” relates to a specific antibody format. This format is particularly stable and can be expressed with high yield in plant cells and plants. scFv-Fc constructs are for example disclosed in Bujak et al (2014), the content of which is incorporated herein by reference. scFv-Fc constructs are dimeric constructs comprising two chains associated to one another for example by one or more disulfide bonds, wherein each of which consist of a structure as follows (in N-C direction):

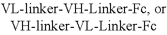

with VL being the variable domain of the light chain of an antibody, VH being the variable domain of the heavy chain of an antibody, and Fc being the constant domain of an antibody.

As used herein, the terms “Fc-VH-VL” and Fc-VH-VL″ relate to a specific antibody format where the VH dmaim is fused to one arm of the Fc domain (CH2 and CH3) and the VH domain is fused to the other arm of the Fc domain.

The use of a full-length IgG-shaped antibody or a scFv-Fc binding domain, or another format comprising an Fc domain, or at least one of CH2 and CH3, confers a longer half-life to the conjugate. Moreover, the Fc part of the antibody might be of utmost importance when CDC (Complement dependent cytotoxicity) or ADCC (Antibody dependent cellular cytotoxicity) activation is required.

Modified antibody formats are for example bi- or trispecific antibody constructs, antibody-based fusion proteins, immunoconjugates and the like. These types are well described in literature and can be used by the skilled person on the basis of the present disclosure, with adding further inventive activity. Furthermore, also monovalent antibodies have been previously described in US 2004/0033561 A1 (referred to therein as monobodies) or WO2007048037; both of which are incorporated herein by reference.

Antibody mimetics are organic compounds—in most cases recombinant proteins or peptides—that, like antibodies, can specifically bind antigens, but that are not structurally related to antibodies. Common advantages over antibodies are better solubility, tissue penetration, stability towards heat and enzymes, and comparatively low production costs. Antibody mimetics are being developed as therapeutic and diagnostic agents, and encompass, inter alia, Affibody molecules, Affilins, Ubiquitins, Affimers, Affitins, Alphabodies, Anticalins, Avimers, DARPins, Fynomers, Kunitz domain peptides, Monobodies and nanoCLAMPs. Antibody mimetics are discussed in great detail, inter alia, in Gebauer and Skerra (2009), incorporated herein by reference.

Generally, the protein binder may consist of a single chain. This is the case, e.g., where the protein binder is a scFv antibody, or a scFv-Fc. In this case, the entire protein binder may be encoded on a single polynucleotide.

In another embodiment the protein binder may comprise two or more chains, like e.g. in a full size IgG or in a F (ab) 2 fragment. In such case it may be provided that the nucleic acid construct may comprise two or more polynucleotides encoding for the different chains or domains for the protein binder.

As used herein, the term “plant” (including the cells derived therefrom) relates to algae (including Chlorophyta and Charophyta/Streptophyta, as well as Mesostigmatophyceae, Chlorokybophyceae and Spirotaenia), and also to land plants (Embryophytes), including Gymnospertms and Angiosperms, including Mono- and Dicotyledonae.

As used herein, the term “transient expression” relates to the temporary expression of genes that are expressed for a short time after a nucleic acid, most frequently plasmid DNA encoding an expression cassette, has been introduced into the host cells or plants.

As used herein, the term “stable expression” relates to expression of genes that are expressed continuously in time after a nucleic acid, most frequently plasmid DNA encoding an expression cassette, has been introduced into the host cells' genome (nuclear or plastid integration). In stably transfected cells, the foreign gene becomes part of the genome and is therefore replicated.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′-3′.

The examples are based on experiments made with Bryodin, as well as for some other toxins belonging to the family of ribosome inactivating proteins. However, the experimental protocols apply for other RIP type I toxins as well, as well as for other configurations.

TABLE 5
non-limiting list of embodiments of binder-toxin fusion
proteins according to an embodiment of the invention

| | | | | | | | substitutions | experiments |
|---|---|---|---|---|---|---|---|---|
| ATB | | | | | | Antibody | in toxin for | shown in |
| NO | description | RIP | target | Antibody | Linker | format | deglycosylation | FIG. |
| 372 | CD79b | n/a | CD79b | pol | n/a | IgG | | 7 |
| | (polatuzumab) | | | | | | | |
| | [HC × LC] | | | | | | | |
| 639 | CD79b | BD1 | CD79b | pol | G4S | IgG | | 3, 7, 8, 12 |
| | (polatuzumab) | | | | | | | |
| | [HC-G4S- | | | | | | | |
| | BD1 × LC] | | | | | | | |
| 655 | CD79b | TRI | CD79b | pol | G4S | IgG | N192S | 4, 7, 12 |
| | (polatuzumab) | | | | | | | |
| | [HC-G4S- | | | | | | | |
| | TRI × LC] | | | | | | | |
| 656 | CD79b | KAR | CD79b | pol | G4S | IgG | S229D | 7, 12 |
| | (polatuzumab) | | | | | | | |
| | [HC-G4S- | | | | | | | |
| | KAR × LC] | | | | | | | |
| 657 | CD79b | MOM | CD79b | pol | G4S | IgG | | 5, 7, 12 |
| | (polatuzumab) | | | | | | | |
| | [HC-G4S- | | | | | | | |
| | MOM × LC] | | | | | | | |
| 659 | CD79b | CUC | CD79b | pol | G4S | IgG | N192S/A228V/ | 7, 12 |
| | (polatuzumab) | | | | | | S229D/R230G | |
| | [HC-G4S- | | | | | | | |
| | CUC × LC] | | | | | | | |
| 660 | CD79b | BD1 | CD79b | pol | G4S | IgG | N192S/S229G | 8 |
| | (polatuzumab) | | | | | | | |
| | [HC-G4S-BD1 | | | | | | | |
| | (N192S) × LC] | | | | | | | |
| 662 | CD79b | BD1 | CD79b | pol | G4S | IgG | N192S/S229D | 12 |
| | (polatuzumab) | | | | | | | |
| | [HC-G4S- | | | | | | | |
| | BD1 (N192S)/ | | | | | | | |
| | (S229D) × LC] | | | | | | | |
| 673 | CD22 | n/a | CD22 | ino | n/a | IgG | | 9, 10 |
| | (inotuzumab) | | | | | | | |
| | [HC × LC] | | | | | | | |
| 678 | CD22 | BD1 | CD22 | ino | G4S | IgG | | 9 |
| | (inotuzumab) | | | | | | | |
| | [HC-G4S-BD1 × | | | | | | | |
| | LC] | | | | | | | |
| 679 | CD22 | BD1 | CD22 | ino | Liopt | IgG | | 9, 10 |
| | (inotuzumab) | | | | | | | |
| | [HC-liopt-BD1 × | | | | | | | |
| | LC] | | | | | | | |
| 752 | CD79b | MOM | CD79b | pol | G4S | IgG | T252D | 12 |
| | (polatuzumab) | | | | | | | |
| | [HC-G4S- | | | | | | | |
| | MOM | | | | | | | |
| | (T252D) × LC] | | | | | | | |
| 1034 | anti tumor | MOM | tumor | n/a | G4S | IgG | T252G | 11 |
| | target AB | | target | | | | | |
| | [HC-G4S- | | | | | | | |
| | MOM(T252G) × | | | | | | | |
| | LC] | | | | | | | |
| 780 | CD79b | Ricin | CD79B | pol | G4S | IgG | | 3 |
| | (polatuzumab) | | | | | | | |
| | [HC-G4S- | | | | | | | |
| | Ricin × LC] | | | | | | | |

*pol = polatuzumab, huSN8 = humanized SN8, ino = inotuzumab

### Materials and Methods

### Genetic Construct Binder Toxin Fusion

Full length Rituximab HC and LC sequences have been used to develop mAb based binder-toxin fusion proteins. Variable parts sequences of the heavy and light chains of rituximab sequences have been assembled in a single chain scFv and fused to a human IgG1 Fc part sequence. A human furin cleavage sequence was then used to fuse the bryodin sequence at the C-terminal part of the LC or the HC of the full-length rituximab or to the C-terminal part of the scFv-Fc to obtain HC+LC-FCS-bryodin, HC-FCS-bryodin+LC and scFv-c-FCS-bryodin fusion proteins sequences. These sequences were produced by gene synthesis flanked with XbaI and Iscel.

A undisclosed plasmid was used to insert ORF coding for binder toxin fusion.

### Genetic Construct Comprising an Antibody

Full length HC and LC antibody sequences have been used to develop antibody-based binder-toxin fusion proteins. Variable parts sequences of the heavy and light chains of the undisclosed sequences have been assembled in a single chain scFv and fused to a human IgG1 Fc part sequence. A human furin cleavage sequence was then respectively used to fuse the human Bryodin sequence at the C-terminal part of the LC or the HC or both of the full-length undisclosed antibody or to the C-terminal part of the scFv-Fc to obtain HC+LC-FCS-Bryodin, HC-FCS-Bryodin+LC, and scFv-Fc-FCS-Bryodin or scFv-Fc-Bryodin fusion proteins sequences. Another binder-toxin fusion protein was realized with scFv-Fc, HC and LC part linked to Bryodin without cleavage site to obtain scFv-Fc-Bryodin, HC+LC-Bryodin, HC-Bryodin+LC, LC-Bryodin+HC-Bryodin. These sequences were produced by gene synthesis flanked with XbaI and Iscel.

Transient Expression in Nicotiana benthamiana Plant Leaves

Nicotiana benthamiana grown under 16 h light/8 h darkness photocycle, 22+/−3° C. 7-8 weeks old plants leaves were transiently transformed by syringe infiltration. Agrobacterium tumefaciens GV3101 (pMP90) harboring the undisclosed plasmid containing genetic construct reaching an 600 nm optical density (OD₆₀₀) around 0.8-1.0 were collected by centrifugation at 3500 g for 10 min. Eventually, bacteria were adjusted to an OD₆₀₀ of 0.5 in infiltration buffer (10 mM MgCl2, 10 mM MES, 100 UM acetosyringone, pH 5.6) and the mixture was infiltrated using a needless syringe. Infiltrated regions were harvested 4 and 6 days post agroinfiltration. Entire leaves harvested 4 days post agroinfiltration were used for protein A purification.

Expression in N. tabacum Cells

Nicotiana tabacum plant suspension cells were grown 5 days at 130 rpm, 25° C. in plant culture media as described by Nagata et al. (1992), the content of which is incorporated herein. Agrobacterium tumefaciens LBA4404 (pBBR1MCS-5.virGN54D) harboring the pPZP-ATB binary plasmids reaching an 600 nm optical density (OD₆₀₀) around 0.8-1.0 were collected by centrifugation at 2000 g for 5 min. Plant cells and bacterial cells were then cocultivated in cocultivation media for 30 min before a 2000 g 5 min centrifugation. After supernatant removal, cells were plated on solid cocultivation media for two days. In the case of transient transformation, cells were then collected and washed three times and cultivated in plant cultivation media containing Cefotaxim and Carbeniclin before being harvested for further analysis. In the case of stable transformation, after the 2 days of solid cocultivation, cells were washed and plated on plant media containing selective kanamycin and Cefotaxim and Carbeniclin antibiotics. Callus were selected 4 weeks later and subcultured on solid media or in liquid suspension cultures for subsequent analysis.

### Protein Analysis: ELISA, SDS-PAGE and Westernblot

Collected leaves tissues (5 g 120 mg) were ground in 400 10 mμL extraction buffer (Tris 0.1M, NaCl 460 mM, EDTA 5 mM, Sodium metabisulfite 5 mM, pH 7.5250 mM Sorbitol, 60 mM Tris, Na2EDTA, 0.6% Polyclar AT, pH8.0). Homogenized tissue was centrifugated at 4° C. for 540 min at 18200 g 40,000 g. Supernatant was then recovered, and the binder-toxin fuion proteins was purified with AmMag™ Protein A Magnetic Beads (L00695; genescript) froze in liquid nitrogen and stored at −20° C. Elution was performed with Glycine 0.1 M, 460 mM NaCl, pH 3.0 and neutralize with 10% Tris 1 M, pH 8.0. The purified binder-toxin proteins were visualized by SDS-PAGE.

Extracted tissue were analyzed by westernblotting. Proteins were boiled for 5 min in reducing or non-reducing SDS loading buffer (80 mM Tris-HCl, pH 6.8, 2% SDS, 10% glycerol, 0.005% bromophenol blue), centrifuged for 5 min at 13 000 rpm and separated by SDS-PAGE (4-20% polyacrylamide). For Western blotting, proteins were electrotransferred onto a PVDF membrane (Biorad) using a semi-dry electrophoretic device (Biorad Trans-Blot Turbo); then, the membrane was blocked for 1 h at room temperature with 3% (w/v) non-fat milk powder in TBST buffer (50 mM Tris-HCl, 150 mM NaCl, 0.5% Tween 20, pH 7.5) and then incubated (TBS-Tween 0.1%+0.5% non-fat dry milk) for 1 h at room temperature with HRP-conjugated antibodies against the anti-human IgG Fc specific region (A0170; Sigma-Aldrich), at a dilution of 1:10.000 or against Bryodin primary antibody from Santa Cruz at a dilution of 1:10.000. The anti-bryodin antibody was followed by HRP-conjugated anti-rabbit antibodies (0545; Sigma), at a dilution of 1:10 000. Proteins were detected by enhanced chemiluminescence (Amersham Imager 600/GE; GE Healthcare).

### Anti CD20 ELISA

For anti-CD20 conjugate specificity analysis, plant extracts were analyzed by 96 well microplate (Greiner) were coated with 100 μL 5 μg/mL CD20 (AcroBiosystems) for 2 h at 37° C. then washed 5 times in washing buffer (TBS Tween 0, 1%). Blocking was then performed with 200 μL BSA 1% in TBS pH8.0 for 30 min at RT then washed 5 times. 100 μL Anti-CD20 control antibody was loaded to realize a calibration curve between 5 and 0 μg/mL and 100 μL samples were loaded on the same 96 well plates for comparison for 2 h at RT then washed 5 times. 100 UL of 1/150.000 diluted detection antibody (goat anti-human HRPO, Bethyl) was loaded and incubated 1 h at RT. Revelation was then performed with 100 μL TMB reaction buffer (Zentech) for 30 min and finally stopper with H₃PO₄ 1M. Enzymatic activity was then analyzed by spectrometry at 450 nm.

### Further ELISA

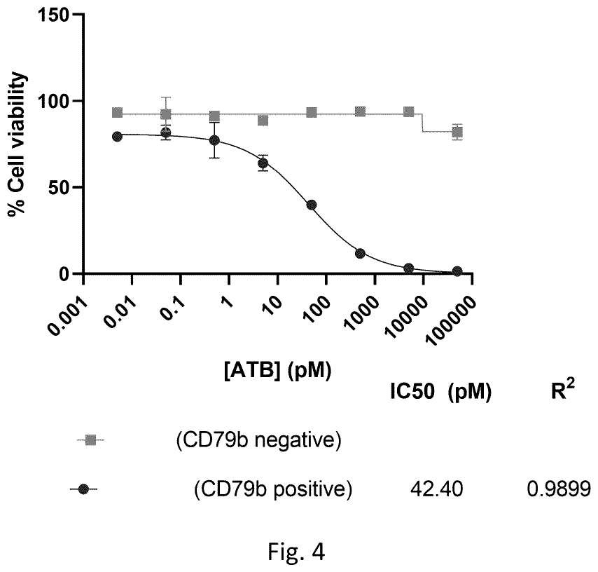

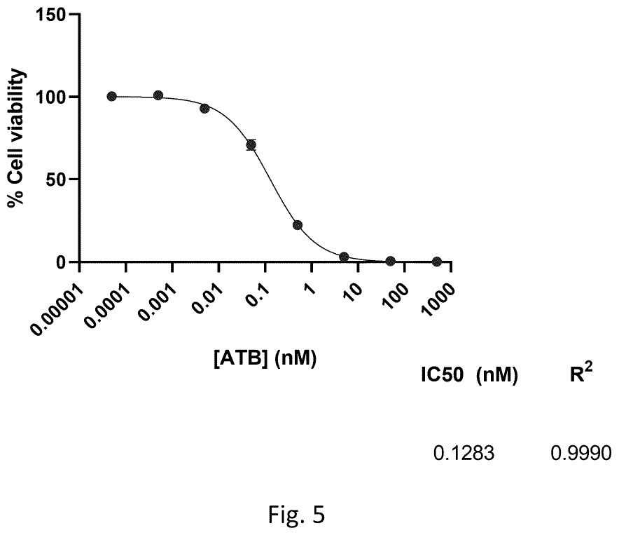

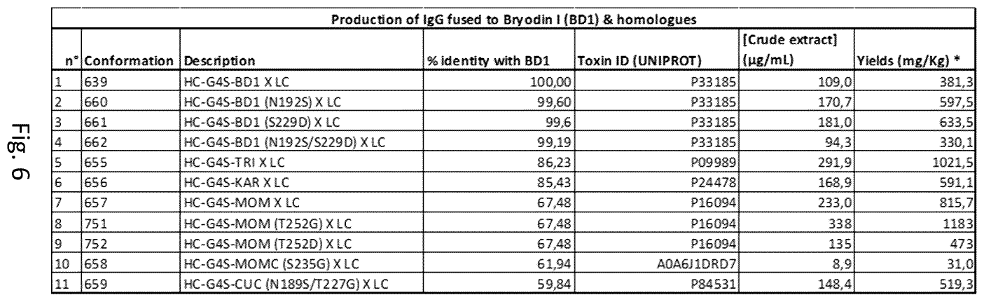

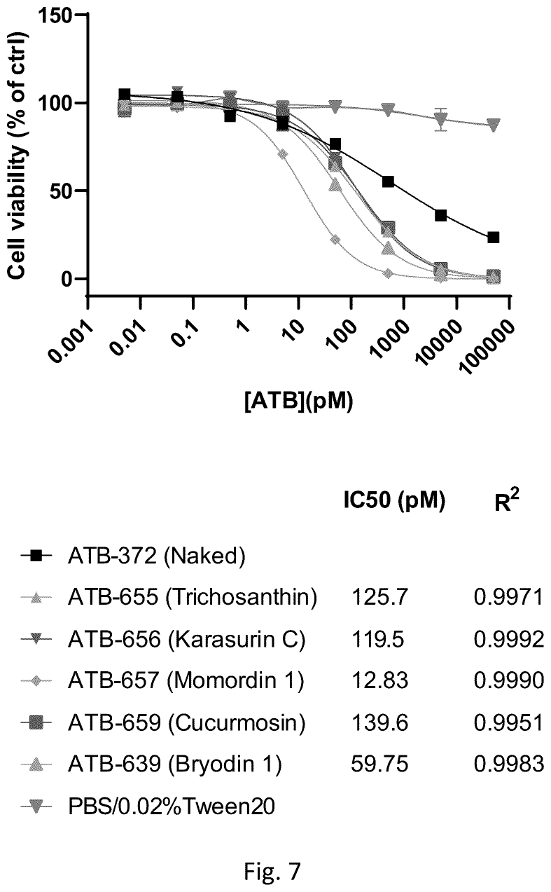

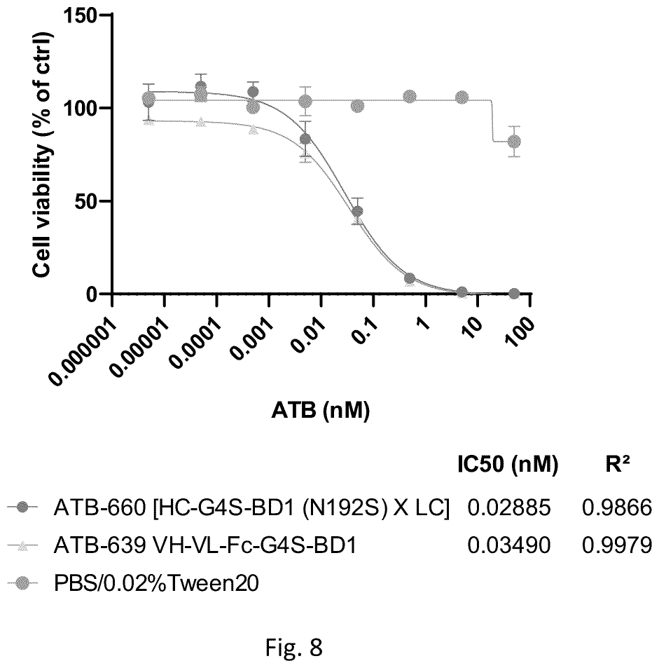

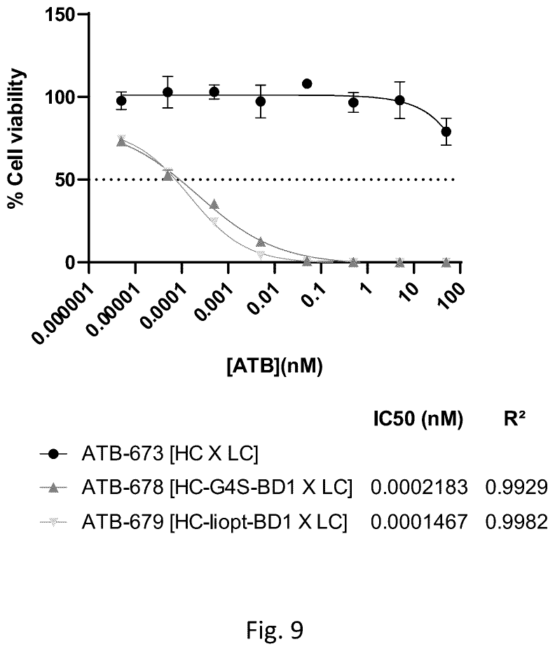

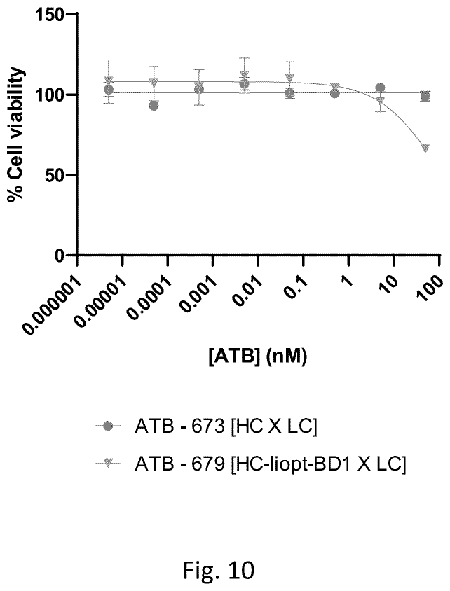

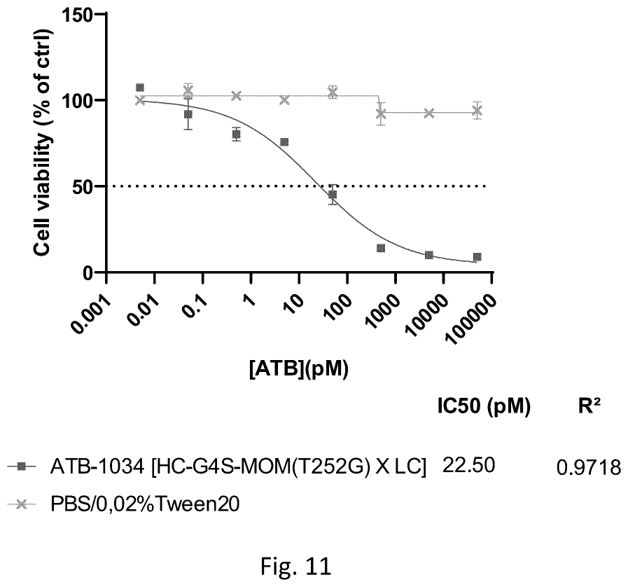

For specificity analysis of a conjugate specific for a non-disclosed antigen (structure expressed on the surface of human cells, and overexpressed in some cancers, called antigen X herein), a purified binder-toxin fusion protein comprising a binder against X was analysed by 96 well microplate (Greiner). The wells were coated with 50 μl of antigen X (2.5 μg/mL) for 1 h at 37° C. then washed 5 times with 250 μL washing buffer (PBS Tween 0.1%). Blocking was then performed with 150 μL hydrocasein (3.6%) in PBST for 30 min at RT then washed 5 times. 50 μL anti antigen control antibody was loaded to realize a calibration curve between 5 and 0 μg/mL and 50 μL samples were loaded on the same 96 well plates for comparison for 1 h at RT then washed 5 times. 50 μL of 1/200.000 diluted detection antibody (goat anti-human HRPO, Bethyl) was loaded and incubated 1 h at RT. Revelation was then performed with 50 μL TMB reaction buffer (Zentech) for 15 min and finally stop with H₃PO₄ 1M. Enzymatic activity was then analyzed by spectrometry at 450 nm. Results are shown in FIG. 4B.

### Protein A Purification

Four days post agroinfiltration, leaves were collected, weighted and grinded in a blender using 2 mL of extraction buffer (TRIS 0.1 M, NaCl 460 mM, EDTA 5 mM, Sodium metabisulfite 5 mM pH 7.5 per gram of fresh agroinfiltrated leaves. The mixture was then filtered through a double Miracloth (Millipore) layer. The filtrate was then centrifugated at 4° C. for 10 min at 40.000 g. Supernatant was then loaded onto protein A resin preequilibrated with washing buffer. Resin was then washed with 10 column volume of 60 mM TRIS 25 mM, 460 mM NaCl pH 7.5. and elution was performed using 100 mM glycine, 460 mM NaCl pH3.0 directly buffered with 10% Tris 1M pH8.0. Enriched protein fractions were then collected, dialyzed and freeze in liquid nitrogen.

### In Vitro Cytotoxicity Assay

The effect of the binder-toxin fusion proteins on the viability of cell lines expressing CD20, CD22 or CD79b was assessed using the Cell Titer Glo Assay (Promega, G9241). In this assay, mono-oxygenation of luciferin is catalyzed by luciferase in presence of Mg 2+ and ATP. This reaction generates a luminescent signal proportional to the number of viable cells.

Depending on the cell line tested, cells were seeded in the cavities of a 96-well plate at a density of 2000 or 5.000 cells/well in 50 μl of growth medium (RPMI1640). Serial dilutions of binder toxin fusion were prepared by adding 10 μl of binder toxin fusion or buffer (PBS, Tween 0.02%) to 40 μl of growth medium. The mixture was added to the cells and incubated for 72 hours at 37° C. with 5% CO2. Binder toxin fusion were tested in duplicate. Buffer served as a negative control, medium and cells only served as blank and untreated control, respectively.

After 72 hours, plates were equilibrated at room temperature for 30 minutes and 100 μl of CellTiter Glo reagent were added to each well. The plates were subsequently placed on a shaking platform for 2 minutes then signal was allowed to stabilize for 10 minutes at room temperature in the dark. Luminescence was then recorded.

To determine the percentage of viability, the average luminescence signal of the blanks (growth medium only) was subtracted from each well and average luminescence signal of untreated cells was set as 100% viability. The average signal of treated cells was then normalized and plotted as a function of the ATB concentration.

The anti-CD20 based binder-toxin fusion proteins were evaluated on target cells (CD20+) and non-target cells K562 (CD20−).

The anti-CD22 based binder-toxin fusion proteins were evaluated on target cells (CD22+) and non-target cells K562 (CD22−).

The anti-CD79b based binder-toxin fusion proteins were evaluated on target cells (CD79+) and non-target cells K562 and LOUCY (CD79−).

### Internalization and Caspase Assay: IncuCyte

To visualize the internalization, caspase activation and cell proliferation of ATB-treated cells, 10,000 cells in 100 μl of growth medium (RPMI1640) were seeded in the cavities of poly-L-ornithine coated plates (96-well). The Incucyte® Caspase-3/7 Dye for Apoptosis Green reagent contains an oligopeptide cleavage sequence (DEVD) conjugated to a DNA-binding dye. The green reagent labels apoptotic cells once the sequence is cleaved by caspase-3/7. Immediately after seeding, cells were supplemented with Incucyte® Caspase-3/7 Dye for Apoptosis Green reagent at a final concentration of 5 μM.

Visualization of internalization of the ATBs require labelling of the ATBs (Fc part) with Incucyte® Human Fabfluor-pH Antibody Labeling Dye for Antibody Internalization at a molar ratio of 1:3. Following conjugation, when labeled antibodies are added to cells, a signal is quickly observed as the Fab-Ab complex is internalized and processed via acidic lyosomes and endosomes resulting in a red fluorescence emission. ATBs were diluted in RPMI in presence of Incucyte® Human Fabfluor-pH Antibody Labeling Dye (1:3 molar ratio) and conjugation was allowed for 15 minutes at 37° C. in the dark. Following conjugation, 100 μl of conjugated ATBs were added to the wells (final ATB concentration: 30 nM) and immediately placed in the incubator. Cell proliferation was determined by phase contrast

Four images were captured per well every 10 to 15 minutes for 4 to 8 hours and then every 4 hours for 72 hours in the green channels. Following subtraction of background signals, red and green signals were quantified

### Peptide Glycoform Analysis

Background on methods for analyzing peptide glycoforms is provided in WO2020169620, the content of which is incorporated herein for enablement purposes.

### Cleavage Assays

Cleavability allowing the release of the toxin have been proved in vitro after addition of recombinant furin on purified HC-FCS-Bryodin+LC (binder-toxin fusion protein). The reaction was performed during 30 min, 2 h and 5 h at 37 degree following addition of 1 μl of 25 units/ml furin (NEB P8077S) to microgram of binder-toxin fusion protein into 15 μl of cleavage buffer (Sodium Acetate 1M pH 5.5+10 mM CaCl2). Cleavage have been visualized by SDS Page Coomassie blue gel (4-20% polyacrylamide).

### Sequence Similarity

Bryodin I (BD1) is a RIP (type I) that possesses a N-glycosidase activity. The protein has two N-glycosylation sites. The percentage of sequence identity with Trichosanthin (TRI), Karasurin (KAR), momordin I (MOM), MOMC, cucurmosin (CUC) was evaluated (FIGS. 2A-2C). Trichosanthin, a naturally non-glycosylated homologues share the highest percentage of identity with BD1 (86.23%). Karasurin has a very close sequence to trichosantin (97.57% identity) and is also non-glycosylated. Momordin shares 67.48% identity with BD1 and possesses one N-glycan site. The WT CUC have two N-glycans sites and the modified sequence CUC (N189S/T227G) was directly evaluated which share 59, 84% with BD1.

### Assays

Binder-toxin fusion proteins based on full length mAb have been constructed with Bryodin, Momordin and trichosantin: HC+LC-Bryodin, HC-Bryodin or LC, HC-Bryodin+LC Bryodin, LC+HC-FCS-Bryodin or Momordin or trichosantin. Unconjugated mAb alone was also constructed as control.

### Cell Viability Assay

Purified binder toxin fusions have been evaluated on cancer cell line for they cytotoxicity. All binder toxin fusions have shown to impair positive cell line viability. Moreover, we demonstrated superiority of type I based binder toxin fusion over a RIP type II based binder toxin fusion that target the same antigen. In addition, binder toxin fusion proteins described above have shown very low effect on target negative cell lines.

Binder fusion toxin are harmless on primary cells HUVEC and HEPG2, constating to high efficacy on cancer cells.

### Assay to Measure Protein Synthesis Inhibition in a Cell Lysate

The Thermo Scientific 1-Step Human Coupled IVT Kit from Thermofischer was used. Briefly, all the components of the reaction are added (Hela lysate, reaction mix, pCFE-GFP-DNA) and ATB or vehicle. The 25-μL reaction mix is transferred in a 384-well plates and fluorescence is measured at 480ex/520em during 90 minutes at 37° C. The GFP fluorescence values are plotted on a graph and then converted in a slope. The slope are then normalized to the reaction mix containing the vehicle (100% of protein synthesis) and the reaction mix containing no pCFE-GFP-DNA and the vehicle (0% protein synthesis).

### RIP Type I Expression Assay

N. benthamiana five weeks-old plants were infiltrated with a suspension of A. tumefaciens. After five days, plants were harvested and homogenized with a ratio of 2:1 extraction buffer volume/weight of leaves. The extract was clarified by centrifugation at 40,000 g for 5 minutes and SPR (Biacore) measurement was performed in order to evaluate the content of expressed proteins in the crude extract.

### PK Evaluation in Mouse Model

Pharmacokinetics has been evaluated in immunocompetent mouse female C57BL/6, age of 7-12 wks old, 20-25 g. Protein toxin fusion proteins were injected via intravenous (tail vein) at dose level of 20 mg/kg. Blood samples (20 μL) are collected by sampling time at—Pre-dose, 30 min, 4 hrs, 8 hrs, 24 hrs (1 day), 48 hrs (2 days), 72 hrs (3 days) and 96 hrs (4 days). Blood is collected into tubes containing K2-EDTA and plasma is isolated from blood by centrifugation at 1,500×g for 10 minutes. Plasma is then transferred to sterile cryovials, aliquoted, and stored at −80° C. until analysis.

The concentration of protein toxin fusion proteins has been determined by sandwich ELISA. This experiment is an ELISA in which the targeted protein (CD79b) is coated on a microplate. Protein toxin fusion proteins are diluted in mouse plasma and detected a secondary antibody against Rabbit-IgG-Fc HRP conjugated. The sandwich ELISA is commonly used for PK evaluation of biologics in animal model and well explained in the review done by Stephanie D. (2013).

Experimental results are summarized in the following table:

TABLE 6
Summary of experimental results

| | | Protein | | | | | |
|---|---|---|---|---|---|---|---|
| | type of | binder | antibody | | | | |
| FIG. | experiment | target | format | linker | toxin | Cells | Finding |
| 3 | viability | CD79b | Full (IgG) | G4S | Bryodin | CD79b+ | Bryodin (RIP I) |
| | assay | | | | or Ricin | | based binder-toxin |
| | | | | | | | fusion protein |
| | | | | | | | reduces cell viability |
| | | | | | | | in a dose dependent |
| | | | | | | | way on target |
| | | | | | | | positive cell line with |
| | | | | | | | an IC50 of 18.37 pM |
| | | | | | | | while chain A ricin |
| | | | | | | | (RIP II) based |
| | | | | | | | binder-toxin fusion |
| | | | | | | | protein only reduced |
| | | | | | | | cell viability with |
| | | | | | | | IC50 of 1.58 nM. |
| | | | | | | | RIP I based binder- |
| | | | | | | | toxin fusion protein |
| | | | | | | | hence has a 88 fold |
| | | | | | | | better potency |
| | | | | | | | compared to RIP II A |
| | | | | | | | chain. This is |
| | | | | | | | surprising because |
| | | | | | | | generally, RIPII are |
| | | | | | | | deemed to be more |
| | | | | | | | toxic than RIPI |
| 4 | viability | CD79b | Full (IgG) | G4S | Tricosanthin | CD79b+ | RIP I tricosanthin |
| | assay | | | | | CD79b− | based binder-toxin |
| | | | | | | | fusion protein |
| | | | | | | | reduces cell viability |
| | | | | | | | in a dose dependent |
| | | | | | | | way on target |
| | | | | | | | positive cell line with |
| | | | | | | | an IC50 of 42.40 |
| | | | | | | | pM, while no IC50 |
| | | | | | | | can be calculated for |
| | | | | | | | antibody target |
| | | | | | | | negative cell line |
| 5 | viability | CD79b | Full (IgG) | \|G4S | Momordin | CD79b+ | RIP I momordin |
| | assay | | | | | | based binder-toxin |
| | | | | | | | fusion protein |
| | | | | | | | reduces cell viability |
| | | | | | | | in a dose dependent |
| | | | | | | | way on target |
| | | | | | | | positive cell line with |
| | | | | | | | an IC50 of 123 pM |
| 6 | Expression | CD79b | Full (IgG) | G4S | Different | | All binder toxin |
| | assay | | | | RIP I | | fusion proteins have |
| | | | | | | | been expressed with |
| | | | | | | | high yields in |
| | | | | | | | Nicotinia |
| | | | | | | | benthamiana |
| 7 | Cell killing | CD79b | Full (IgG) | G4S | Different | CD79b+ | All binder toxin |
| | assay | | | | RIP I | | fusion proteins with |
| | | | | | | | the different RIP |
| | | | | | | | Type I have |
| | | | | | | | extremely high cell |
| | | | | | | | killing potency |
| 8 | Cell killing | CD79b | | G4S | Bryodin | | Deglycosylation of |
| | assay | | | | | | the toxin does not |
| | | | | | | | affect potency of the |
| | | | | | | | binder toxin fusion |
| | | | | | | | protein |
| 9 | Cell killing | CD22 | Full (IgG) | G4S | Bryodin | CD22+ | Liopt is a furin |
| | assay | | | vs | | (mammalian) | cleavable linker that |
| | | | | liopt | | | is cleaved by |
| | | | | | | | mammalian cells |
| | | | | | | | while G4S is non |
| | | | | | | | cleavable. Linker |
| | | | | | | | cleavability does yet |
| | | | | | | | not affect potency of |
| | | | | | | | the binder toxin |
| | | | | | | | fusion protein in this |
| | | | | | | | cell assay |
| 10 | Cell killing | CD22 | Full (IgG) | liopt | Bryodin | CD22− | Binder-toxin fusion |
| | assay | | | | | | protein comprising |
| | | | | | | | bryodin with the liopt |
| | | | | | | | linker and an anti |
| | | | | | | | CD22 antibody is |
| | | | | | | | not toxic to target |
| | | | | | | | negative cell line |
| 11 | Cell killing | Cancer | Full (IgG) | G4S | Momordin | Anti cancer | Binder-toxin fusion |
| | assay | antigen | | | | antigen | protein comprising |
| | | | | | | | momordin is also |
| | | | | | | | toxic in context with |
| | | | | | | | other cancer |
| | | | | | | | targets/anti cancer |
| | | | | | | | target antibodies |
| 12 | Protein | CD79b | Full (IgG) | | Different | n/a | Protein synthesis |
| | synthesis | | | | RIP I | | inhibition in HELA |
| | inhibition | | | | | | cell lysate. Complete |
| | assay | | | | | | binder-toxin fusion |
| | | | | | | | proteins inhibit |
| | | | | | | | protein synthesis |
| | | | | | | | even target- |
| | | | | | | | independently |
| 13 | Internalization | CD79b | Full (IgG) | | Bryodin | CD79b+ | The binder toxin |
| | assay | | | | | | fusion protein |
| | | | | | | | comprising BD1 is |
| | | | | | | | much better |
| | | | | | | | internalized than the |
| | | | | | | | naked antibody |
| 15 | PK assay | CD79b | Full (IgG) | | Bryodin/ | | Deglycosylation |
| | | | | | Momordin | | increases serum half |
| | | | | | | | life. |

### REFERENCES

The content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.
Léa V. Zinsli, Noël Stierlin, Martin J. Loessner, Mathias Schmelcher, Deimmunization of protein therapeutics-Recent advances in experimental and computational epitope prediction and deletion, Computational and Structural Biotechnology Journal, Volume 19, 2021, Pages 315-329
Vivanco, J. M.; Savary, B. J.; Flores, H. E. Characterization of two novel type i ribosome-inactivating proteins from the storage roots of the andean crop Mirabilis expansa. Plant Physiol. 1999, 119, 1447-1456.
Wilbers, R. H. P., Westerhof, L. B., van Raaij, D. R., van Adrichem, M., Prakasa, A. D., Lozano-Torres, J. L., Bakker, J., Smant, G. and Schots, A. (2016), Co-expression of the protease furin in Nicotiana benthamiana leads to efficient processing of latent transforming growth factor-β1 into a biologically active protein. Plant Biotechnol J, 14:1695-1704.
Lim S H, Beers S A, French R R, Johnson P W, Glennie M J, Cragg M S. Anti-CD20 monoclonal antibodies: historical and future perspectives. Haematologica. 2010 January; 95 (1): 135-43.
Reff M E, Carner K, Chambers K S, Chinn P C, Leonard J E, Raab R, et al. Depletion of B cells in vivo by a chimeric mouse human monoclonal antibody to CD20. Blood. 1994; 83 (2): 435-45.
Hagenbeek A, Gadeberg O, Johnson P, Pedersen L M, Walewski J, Hellmann A, et al. First clinical use of ofatumumab, a novel fully human anti-CD20 monoclonal antibody in relapsed or refractory follicular lymphoma: results of a phase 1/2 trial. Blood. 2008; 111 (12): 5486-95
Umana P, Moessner E, Bruenker P, Unsin G, Puentener U, Suter T, et al. Novel 3rd Generation Humanized Type II CD20 Antibody with Glycoengineered Fc and Modified Elbow Hinge for Enhanced ADCC and Superior Apoptosis Induction. Blood. 2006; 108 abstr.229.
Milani C, Castillo J. Veltuzumab, an anti-CD20 mAb for the treatment of non-Hodgkin's lymphoma, chronic lymphocytic leukemia and immune thrombocytopeniarpura. Curr Opin Mol Ther. 2009 April; 11 (2): 200-7.
Genovese, Mark & Kaine, Jeffrey & Lowenstein, Mitchell & Giudice, José & Baldassare, Andrew & Schechtman, Joy & Fudman, Edward & Kohen, Michael & Gujrathi, Sheila & Trapp, Robert & Sweiss, Nadera & Spaniolo, Greg & Dummer, Wolfgang & Arthur, E. & Baldassare, A. & Boling, E. & Booth, J. & Caldron, Paul & Carfagno, D. & Wolfe, S. (2008). Ocrelizumab, a humanized anti-CD20 monoclonal antibody, in the treatment of patients with rheumatoid arthritis: A phase I/II randomized, blinded, placebo-controlled, dose-ranging study. Arthritis and rheumatism. 58. 2652-61.
Onda M, Beers R, Xiang L, Lee B, Weldon J E, Kreitman R J, Pastan I. Recombinant immunotoxin against B-cell malignancies with no immunogenicity in mice by removal of B-cell epitopes. Proc Natl Acad Sci USA. 2011 Apr. 5; 108 (14): 5742-7.
Joseph Wynne, David Wright, Wendy Stock; Inotuzumab: from preclinical development to success in B-cell acute lymphoblastic leukemia. Blood Adv 2019; 3 (1): 96-104.
Abdel-Ghany S. E. (2015). Engineering of plants for the production of commercially important products: approaches and accomplishments. In Plant biology and biotechnology (pp. 551-577). Springer.
Ali, S., & Kim, W. C. (2019). A fruitful decade using synthetic promoters in the improvement of transgenic plants. Frontiers in plant science, 10.
Francisco J A., et al., Binder-toxin fusion protein chemistry 8.5 (1997): 708-713.
Hellwig, S et al., Nature biotechnology, 22 (11), 1415
Häkkinen S et al., Front Plant Sci. 2018; 9:45.
Shaaltiel, Y et al., (2007) Plant biotechnology journal, 5 (5), 579-590.
Santos R et al., Front. Plant Sci., Front. Plant Sci., 11 Mar. 2016.
Plasson, C et al., (2009), Recombinant Proteins From Plants (pp. 145-161). Humana Press.
Daniell H et al., Trends Plant Sci. 2001 May; 6 (5): 219-226.
Turk V et al, Biochimica et Biophysica Acta (BBA)-Proteins and Proteomics, Volume 1824, Issue 1, January 2012, Pages 68-88
Kumar S et al, PLOS One. 2014; 9 (10): e110539.
Eckard U et al, Matrix Biology, Volume 49, January 2016, Pages 37-60
Rawlings N D, Biochimie, Volume 122, March 2016, Pages 5-30
Schmohl J et al, Toxins (Basel). 2015 October; 7 (10): 4067-4082.
Yehudit Grinberg Y and Benhar I, Biomedicines. 2017 June; 5 (2): 28.
Klaine, S. J. and M A. Lewis. Algal And Plant Toxicity Testing. 1995 Chapter 8, in Hoffman et al (eds), Handbook of Ecotoxicology. Lewis Publishers, Boca Raton, FL, 163-184, (1995).
Köhler and Milstein, Nature. Bd. 256, S. 495-497
Okazaki et al., Blood, 81:84-94 (1993))
Polson et al., Blood. 2007; 110 (2): 616-623
Grawunder, Ulf, and Stefan Barth, eds. Next Generation Antibody Drug Conjugates (ADCs) and Immunotoxins. Springer, 2017
Nagata, T et al., (1992). International Review of Cytology (Vol. 132, pp. 1-30).
Gebauer and Skerra, Curr Opin Chem Biol. 2009 June; 13 (3): 245-55
Krishnan, Rajesh, et al. “Expression of recombinant trichosanthin, a ribosome-inactivating protein, in transgenic tobacco.” Journal of Biotechnology 97.1 (2002): 69-88.
Brinkmann U, Kontermann R E. The making of bispecific antibodies. MAbs. 2017 February/March; 9 (2): 182-212. doi: 10.1080/19420862.2016.1268307.
Bujak E, Matasci M, Neri D, Wulhfard S. Reformatting of scFv antibodies into the scFv-Fc format and their downstream purification. Methods Mol Biol. 2014; 1131:315-34.
Gan, Stephanie D., and Kruti R. Patel. “Enzyme immunoassay and enzyme-linked immunosorbent assay.” J Invest Dermatol 133.9 (2013): e12.

### Sequences

The following sequences form part of the disclosure of the present application. A WIPO ST 26 compatible electronic sequence listing is provided with this application, too. For the avoidance of doubt, if discrepancies exist between the sequences in the following table and the electronic sequence listing, the sequences in this table shall be deemed to be the correct ones.

Note also that in some embodiments, the respective amino acid sequence has or has not a signal peptide/lead peptide. All embodiments shall be deemed to be disclosed together with the signal peptide/lead peptide and without the signal peptide/lead peptide.

TABLE 7
Sequence listing

| No | Qualifier | Sequence |
|---|---|---|
| 1 | Momordin | DVSFRLSGADPRSYGMFIKDLRNALPFREKVYNIPLLLPSVSGAGRYLL |
| | | MHLFNYDGKTITVAVDVTNVYIMGYLADTTSYFFNEPAAELASQYVFRD |
| | | ARRKITLPYSGNYERLQIAAGKPREKIPIGLPALDSAISTLLHYDSTAAAG |
| | | ALLVLIQTTAEAARFKYIEQQIQERAYRDEVPSLATISLENSWSGLSKQIQ |
| | | LAQGNNGIFRTPIVLVDNKGNRVQITNVTSKVVTSNIQLLLNTRNI |
| 2 | Bryodin I | DVSFRLSGATTTSYGVFIKNLREALPYERKVYNIPLLRSSISGSGRYTLLH |
| | | LTNYADETISVAVDVTNVYIMGYLAGDVSYFFNEASATEAAKFVFKDAKK |
| | | KVTLPYSGNYERLQTAAGKIRENIPLGLPALDSAITTLYYYTASSAASALL |
| | | VLIQSTAESARYKFIEQQIGKRVDKTFLPSLATISLENNWSALSKQIQIAST |
| | | NNGQFESPVVLIDGNNQRVSITNASARVVTSNIALLLNRNNIA |
| 3 | Cucurmosin | NVRFDLSSATSSSYKTFIKNLREALPKDGKVYDIPVLLSTVMDSRRFILID |
| | | LVNYDGQSITAAIDVLNVYIVAYSTGTVSYFFQQVPAQAPKLLFKGTQQR |
| | | TLPYTGNYENLQTAAKKLRENIELGLPALDSAITTLFHYNAEAAASALLVL |
| | | IQTTSEAARFRYIELQIANNVGTKFKPSQTIISLENNWSALSKQIQIAKNKN |
| | | GQFETPVILIDPQGNRVQITNVTSNVVTQNIQLLLNIGAT |
| 4 | Bryodin II | VDINFSLIGATGATYKTFIRNLRTKLTVGTPRVYDIPVLRNAAAGLARFQL |
| | | VTLTNYNGESVTVALDVVNVYVVAYRAGNTAYFLADASTEANNVLFAGI |
| | | NHVRLPYGGNYDGLETAAGRISRENIELGFSEISSAIGNMFRHNPGTSV |
| | | PRAFIVIIQTVSEAARFKYIEQRVSENVGTKFKPDPAFLSLQNAWGSLSE |
| | | QIQIAQTRGGEFARPVELRTVSNTPTFVTNVNSPVVKGIALLLYFRVNVG |
| | | TDNVFAMSLSTY |
| 5 | Trichosantin | DVSFRLSGATSSSYGVFISNLRKALPNERKLYDIPLLRSSLPGSQRYALI |
| | | HLTNYADETISVAIDVTNVYIMGYRAGDTSYFFNEASATEAAKYVFKDAM |
| | | RKVTLPYSGNYERLQTAAGKIRENIPLGLPALDSAITTLFYYNANSAASAL |
| | | MVLIQSTSEAARYKFIEQQIGKRVDKTFLPSLAIISLENSWSALSKQIQIAS |
| | | TNNGQFESPVVLINAQNQRVTITNVDAGVVTSNIALLLNRNNMA |
| 6 | Karasurin | EGDVSFRLSGATSSSYGVFISNLRKALPYERKLYDIPLLRSTLPGSQRYA |
| | | LIHLTNYADETISVAIDVTNVYVMGYRAGDTSYFFNEASATEAAKYVFKD |
| | | AKRKVTLPYSGNYERLQIAAGKIRENIPLGLPALDSAITTLFYYNANSAAS |
| | | ALMVLIQSTSEAARYKFIEQQIGKRVDKTFLPSLAIISLENSWSALSKQIQI |
| | | ASTNNGQFETPVVLINAQNQRVTITNVDAGVVTSNIALLLNRNNMA |
| 7 | MOMC | VEGDVSFRLLGATTKSYKDFIANFRNDLASHKRVYNIRLLSLTVPGPSRY |
| | | TLVHLYNYDDQTITLAVDVINVYIMGYRNGHVSYFFNEPAAAEASKFVFK |
| | | DAQRRVTLPFTSNYAGLETAAGKIRERIPLGLPAMNNAITNLVYYDTSPR |
| | | NAIAAAFLVLIQSTAEAARFQYIEKVIGKDVEKDFLPSEALLSLENNWGAL |
| | | SKQIQIAEKNNGKFKDPVLLTDNQGKKVQINNVGSNVVTANIQLLLNYQQ |
| | | QVLAFDQDIMSSELRLGHGSYVTM |
| 8 | ME1 | APPTLATLDLTAAANYPPFITNMRNVLSEKDKNGKDVLLCTMKKISTTVP |
| | | SPRYAYVDIKASATQTVTLAIDRTNTYVLGYRDIFGGTDRAAFFKDVYDD |
| | | AKDLFPDAKGKNRIKLSYGSQYTTLGDRTKVPLGIKSLRISITAIYGEAAG |
| | | TDLDKNRREFFLLALQMVAEATRFKYISDKIPTERDYDTLKVDNHMIALE |
| | | NGWDLLSTAIYNAKSSTTKPTKCELLKTPVSLIWLGQNEWNFNSVEEIAK |
| | | VVGLLKAKGKKLSTNNDDDNNGDDCGSVVVAS |
| 9 | Bryodin | DVSFRLSGATTTSYGVFIKNLREALPYERKVYNIPLLRSSISGSGRYTLLH |
| | (N192S) | LTNYADETISVAVDVTNVYIMGYLAGDVSYFFNEASATEAAKFVFKDAKK |
| | | KVTLPYSGNYERLQTAAGKIRENIPLGLPALDSAITTLYYYTASSAASALL |
| | | VLIQSTAESARYKFIEQQIGKRVDKTFLPSLATISLENNWSALSKQIQIAST |
| | | NNGQFESPVVLIDGNNQRVSITNADARVVTSNIALLLNRNNIA |
| 10 | Bryodin | DVSFRLSGATTTSYGVFIKNLREALPYERKVYNIPLLRSSISGSGRYTLLH |
| | (S229D) | LTNYADETISVAVDVTNVYIMGYLAGDVSYFFNEASATEAAKFVFKDAKK |
| | | KVTLPYSGNYERLQTAAGKIRENIPLGLPALDSAITTLYYYTASSAASALL |
| | | VLIQSTAESARYKFIEQQIGKRVDKTFLPSLATISLENNWSALSKQIQIAST |
| | | NNGQFESPVVLIDGNNQRVSITNADARVVTSNIALLLNRNNIA |
| 11 | Bryodin | DVSFRLSGATTTSYGVFIKNLREALPYERKVYNIPLLRSSISGSGRYTLLH |
| | (N192S/S229D) | LTNYADETISVAVDVTNVYIMGYLAGDVSYFFNEASATEAAKFVFKDAKK |
| | | KVTLPYSGNYERLQTAAGKIRENIPLGLPALDSAITTLYYYTASSAASALL |
| | | VLIQSTAESARYKFIEQQIGKRVDKTFLPSLATISLENSWSALSKQIQIAST |
| | | NNGQFESPVVLIDGNNQRVSITNADARVVTSNIALLLNRNNIA |
| 12 | Momordin | DVSFRLSGADPRSYGMFIKDLRNALPFREKVYNIPLLLPSVSGAGRYLL |
| | (T252G) | MHLFNYDGKTITVAVDVTNVYIMGYLADTTSYFFNEPAAELASQYVFRD |
| | | ARRKITLPYSGNYERLQIAAGKPREKIPIGLPALDSAISTLLHYDSTAAAG |
| | | ALLVLIQTTAEAARFKYIEQQIQERAYRDEVPSLATISLENSWSGLSKQIQ |
| | | LAQGNNGIFRTPIVLVDNKGNRVQITNVGSKVVTSNIQLLLNTRNI |
| 13 | Momordin | DVSFRLSGADPRSYGMFIKDLRNALPFREKVYNIPLLLPSVSGAGRYLL |
| | (T252D) | MHLFNYDGKTITVAVDVTNVYIMGYLADTTSYFFNEPAAELASQYVFRD |
| | | ARRKITLPYSGNYERLQIAAGKPREKIPIGLPALDSAISTLLHYDSTAAAG |
| | | ALLVLIQTTAEAARFKYIEQQIQERAYRDEVPSLATISLENSWSGLSKQIQ |
| | | LAQGNNGIFRTPIVLVDNKGNRVQITNVDSKVVTSNIQLLLNTRNI |
| 14 | Cucurmosin | NVRFDLSSATSSSYKTFIKNLREALPKDGKVYDIPVLLSTVMDSRRFILID |
| | (N189S/T227G) | LVNYDGQSITAAIDVLNVYIVAYSTGTVSYFFQQVPAQAPKLLFKGTQQR |
| | | TLPYTGNYENLQTAAKKLRENIELGLPALDSAITTLFHYNAEAAASALLVL |
| | | IQTTSEAARFRYIELQIANNVGTKFKPSQTIISLENSWSALSKQIQIAKNKN |
| | | GQFETPVILIDPQGNRVQITNVGSNVVTQNIQLLLNIGAT |
| 15 | HC of anti | QVQLQQPGAELVKPGASVKMSCKASGYTFTSYNMHWVKQTPGRGLE |
| | CD20 antibody | WIGAIYPGNGDTSYNQKFKGKATLTADKSSSTAYMQLSSLTSEDSAVYY |
| | C2B8 | CARSTYYGGDWYFNVWGAGTTVTVSAASTKGPSVFPLAPSSKSTSGG |
| | | TAALGCLVKDYFPEPVTVSWNSGALTSGVHTFPAVLQSSGLYSLSSVVT |
| | | VPSSSLGTQTYICNVNHKPSNTKVDKKAEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGK |
| 16 | LC of anti | QIVLSQSPAILSASPGEKVTMTCRASSSVSYIHWFQQKPGSSPKPWIYA |
| | CD20 antibody | TSNLASGVPVRFSGSGSGTSYSLTISRVEAEDAATYYCQQWTSNPPTF |
| | C2B8 | GGGTKLEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| 17 | (anti CD20 | QVQLQQPGAELVKPGASVKMSCKASGYTFTSYNMHWVKQTPGRGLE |
| | antibody | WIGAIYPGNGDTSYNQKFKGKATLTADKSSSTAYMQLSSLTSEDSAVYY |
| | C2B8)-HC- | CARSTYYGGDWYFNVWGAGTTVTVSAASTKGPSVFPLAPSSKSTSGG |
| | FCS-Bryodin | TAALGCLVKDYFPEPVTVSWNSGALTSGVHTFPAVLQSSGLYSLSSVVT |
| | | VPSSSLGTQTYICNVNHKPSNTKVDKKAEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGK |
| | | DVSFRLSGATTTSYGVFIKNLREALPYERKVYNIPLLRSSISGSGRYTLLH |
| | | LTNYADETISVAVDVTNVYIMGYLAGDVSYFFNEASATEAAKFVFKDAKK |
| | | KVTLPYSGNYERLQTAAGKIRENIPLGLPALDSAITTLYYYTASSAASALL |
| | | VLIQSTAESARYKFIEQQIGKRVDKTFLPSLATISLENNWSALSKQIQIAST |
| | | NNGQFESPVVLIDGNNQRVSITNASARVVTSNIALLLNRNNIA |
| 18 | Rituximab | YTFTSYNMH |
| | HCDR1 | |
| 19 | Rituximab | WIGAIYPGNGDTSY |
| | HCDR2 | |
| 20 | Rituximab | RSTYYGGDWYFNV |
| | HCDR3 | |
| 21 | Rituximab | SSVSYIH |
| | LCDR1 | |
| 22 | Rituximab | PWIYATSNLAS |
| | LCDR2 | |
| 23 | Rituximab | QQWTSNPP |
| | LCDR3 | |
| 24 | Glycosylation | EEQYNSTYR |
| | site | |
| 25 | Glycosylation | NFSNDIMLLQLER |
| | site | |
| 26 | Furin | HRRRKRSLDTS |
| | Cleavage site | |
| | (FCS) (= liopt) | |
| 27 | Ricin A chain | EDNNIFPKQYPIINFTTAGATVQSYTNFIRAVRGRLTTGADVRHEIPVLPN |
| | | RVGLPINQRFILVELSNHAELSVTLALDVTNAYVVGYRAGNSAYFFHPDN |
| | | QEDAEAITHLFTDVQNRYTFAFGGNYDRLEQLAGSLRENIELGNGPLEE |
| | | AISALYYYSTGGTQLPTLARSFIVCIQMISEAARFQYIEGEMRTRIRYNRR |
| | | SAPDPSVITLENSWGRLSTAIQESNQGAFASPIQLQRRNGSKFSVYDVSI |
| | | LIPIIALMVYRCAPPP |
| 28 | (anti CD20 | QVQLQQPGAELVKPGASVKMSCKASGYTFTSYNMHWVKQTPGRGLE |
| | antibody | WIGAIYPGNGDTSYNQKFKGKATLTADKSSSTAYMQLSSLTSEDSAVYY |
| | C2B8)-HC- | CARSTYYGGDWYFNVWGAGTTVTVSAASTKGPSVFPLAPSSKSTSGG |
| | FCS- | TAALGCLVKDYFPEPVTVSWNSGALTSGVHTFPAVLQSSGLYSLSSVVT |
| | Momordin | VPSSSLGTQTYICNVNHKPSNTKVDKKAEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGK |
| | | DVSFRLSGADPRSYGMFIKDLRNALPFREKVYNIPLLLPSVSGAGRYLL |
| | | MHLFNYDGKTITVAVDVTNVYIMGYLADTTSYFFNEPAAELASQYVFRD |
| | | ARRKITLPYSGNYERLQIAAGKPREKIPIGLPALDSAISTLLHYDSTAAAG |
| | | ALLVLIQTTAEAARFKYIEQQIQERAYRDEVPSLATISLENSWSGLSKQIQ |
| | | LAQGNNGIFRTPIVLVDNKGNRVQITNVTSKVVTSNIQLLLNTRNI |
| 29 | (anti CD79 | EVQLVESGGGLVQPGGSLRLSCAASGYTFSSYWIEWVRQAPGKGLEW |
| | antibody VH)- | IGEILPGGGDTNYNEIFKGRATFSADTSKNTAYLQMNSLRAEDTAVYYC |
| | (Rituximab | TRRVPIRLDYWGQGTLVTVSS |
| | CH1-CH3)- | ASTKGPSVFPLAPSSKSTSGGTAALGCLVKDYFPEPVTVSWNSGALTS |
| | FCS- | GVHTFPAVLQSSGLYSLSSVVTVPSSSLGTQTYICNVNHKPSNTKVDKK |
| | Momordin | AEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| | | HRRRKRSLDTS |
| | | DVSFRLSGADPRSYGMFIKDLRNALPFREKVYNIPLLLPSVSGAGRYLL |
| | | MHLFNYDGKTITVAVDVTNVYIMGYLADTTSYFFNEPAAELASQYVFRD |
| | | ARRKITLPYSGNYERLQIAAGKPREKIPIGLPALDSAISTLLHYDSTAAAG |
| | | ALLVLIQTTAEAARFKYIEQQIQERAYRDEVPSLATISLENSWSGLSKQIQ |
| | | LAQGNNGIFRTPIVLVDNKGNRVQITNVTSKVVTSNIQLLLNTRNI |
| 30 | (anti CD79 | DIQLTQSPSSLSASVGDRVTITCKASQSVDYEGDSFLNWYQQKPGKAP |
| | antibody VL)- | KLLIYAASNLESGVPSRFSGSGSGTDFTLTISSLQPEDFATYYCQQSNE |
| | (Rituximab | DPLTFGQGTKVEIK |
| | Kappa | RTVAAPSVFIFPPSDEQLKSGTASVVCLLNNFYPREAKVQWKVDNALQS |
| | constant) | GNSQESVTEQDSKDSTYSLSSTLTLSKADYEKHKVYACEVTHQGLSSP |
| | | VTKSFNRGEC |
| 31 | (anti CD20 | QVQLQQPGAELVKPGASVKMSCKASGYTFTSYNMHWVKQTPGRGLE |
| | antibody | WIGAIYPGNGDTSYNQKFKGKATLTADKSSSTAYMQLSSLTSEDSAVYY |
| | C2B8)-HC- | CARSTYYGGDWYFNVWGAGTTVTVSAASTKGPSVFPLAPSSKSTSGG |
| | FCS- | TAALGCLVKDYFPEPVTVSWNSGALTSGVHTFPAVLQSSGLYSLSSVVT |
| | Trichosantin | VPSSSLGTQTYICNVNHKPSNTKVDKKAEPKSCDKTHTCPPCPAPELLG |
| | | GPSVFLFPPKPKDTLMISRTPEVTCVVVDVSHEDPEVKFNWYVDGVEV |
| | | HNAKTKPREEQYNSTYRVVSVLTVLHQDWLNGKEYKCKVSNKALPAPI |
| | | EKTISKAKGQPREPQVYTLPPSRDELTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGKHRRRKRSLDTSDVSFRLSGATSSSYGVFIS |
| | | NLRKALPNERKLYDIPLLRSSLPGSQRYALIHLTNYADETISVAIDVTNVYI |
| | | MGYRAGDTSYFFNEASATEAAKYVFKDAMRKVTLPYSGNYERLQTAAG |
| | | KIRENIPLGLPALDSAITTLFYYNANSAASALMVLIQSTSEAARYKFIEQQI |
| | | GKRVDKTFLPSLAIISLENSWSALSKQIQIASTNNGQFESPVVLINAQNQ |
| | | RVTITNVDAGVVTSNIALLLNRNNMA |
| 32 | (anti CD79 | EVQLVESGGGLVQPGGSLRLSCAASGYTFSSYWIEWVRQAPGKGLEW |
| | antibody VH)- | IGEILPGGGDTNYNEIFKGRATFSADTSKNTAYLQMNSLRAEDTAVYYC |
| | (Rituximab | TRRVPIRLDYWGQGTLVTVSS |
| | CH1-CH3)- | ASTKGPSVFPLAPSSKSTSGGTAALGCLVKDYFPEPVTVSWNSGALTS |
| | FCS- | GVHTFPAVLQSSGLYSLSSVVTVPSSSLGTQTYICNVNHKPSNTKVDKK |
| | Momordin | AEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLH |
| | | QDWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLY |
| | | SKLTVDKSRWQQGNVFSCSVMHEALHNHYTQKSLSLSPG |
| | | HRRRKRSLDTS |
| | | DVSFRLSGADPRSYGMFIKDLRNALPFREKVYNIPLLLPSVSGAGRYLL |
| | | MHLFNYDGKTITVAVDVTNVYIMGYLADTTSYFFNEPAAELASQYVFRD |
| | | ARRKITLPYSGNYERLQIAAGKPREKIPIGLPALDSAISTLLHYDSTAAAG |
| | | ALLVLIQTTAEAARFKYIEQQIQERAYRDEVPSLATISLENSWSGLSKQIQ |
| | | LAQGNNGIFRTPIVLVDNKGNRVQITNVTSKVVTSNIQLLLNTRNI |
| 33 | (anti CD79 | DIQLTQSPSSLSASVGDRVTITCKASQSVDYEGDSFLNWYQQKPGKAP |
| | antibody VL)- | KLLIYAASNLESGVPSRFSGSGSGTDFTLTISSLQPEDFATYYCQQSNE |
| | (Rituximab | DPLTFGQGTKVEIK |
| | Kappa | RTVAAPSVFIFPPSDEQLKSGTASVVCLLNNFYPREAKVQWKVDNALQS |
| | constant) | GNSQESVTEQDSKDSTYSLSSTLTLSKADYEKHKVYACEVTHQGLSSP |
| | | VTKSFNRGEC |
| 34 | anti CD79B | GYTFSSYWIE |
| | HCDR1 | |
| 35 | anti CD79B | GEILPGGGDTNYNEIFKG |
| | HCDR2 | |
| 36 | anti CD79B | TRRVPIRLDY |
| | HCDR3 | |
| 37 | anti CD79B | KASQSVDYEGDSFLN |
| | LCDR1 | |
| 38 | anti CD79B | AASNLES |
| | LCDR2 | |
| 39 | anti CD79B | CQQSNEDPLT |
| | LCDR3 | |
| 40 | G4S linker | GGGGS |
| 41 | DDDDKGGS | DDDDKGGS |
| | linker | |
| 42 | DDDDK linker | DDDDK |
| 43 | Light chain | DVVVTQTPLSLPVSFGDQVSISCRSSQSLANSYGNTFLSWYLHKPGQS |
| | variable | PQLLIYGISNRFSGVPDRFTGSGSGTDFTLKISTIKPEDLGMYYCLQGTH |
| | domain of anti | QPYTFGGGTKLEIKR |
| | CD22 antibody | |
| | inotuzumab | |
| 44 | Heavy chain | EVQLVQSGAEVKKPGASVKVSCKASGYRFTNYWIHWVRQAPGQGLEW |
| | variable | IGGINPGNNYATYRRKFQGRVTMTADTSTSTVYMELSSLRSEDTAVYYC |
| | domain of anti | TREGYGNYGAWFAYWGQGTLVTVSS |
| | CD22 antibody | |
| | inotuzumab | |

As used herein, the terms “general,” “generally,” and “approximately” are intended to account for the inherent degree of variance and imprecision that is often attributed to, and often accompanies, any design and manufacturing process, including engineering tolerances, and without deviation from the relevant functionality and intended outcome, such that mathematical precision and exactitude is not implied and, in some instances, is not possible.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A binder-toxin fusion protein comprising at least
a) a protein binder, and
b) a Ribosome-inactivating protein (RIP) type 1 or an active fragment thereof.

2. The binder-toxin fusion protein according to claim 1, wherein the Ribosome-inactivating protein (RIP) is at least one selected from the group consisting of:
Momordin
Bryodin I
Cucurmosin
Bryodin II
Trichosanthin
Karasurin
MOMC
ME1, and/or
ME2

3. The binder-toxin fusion protein according to claim 1 or 2, wherein the Ribosome-inactivating protein (RIP) comprises at least one amino acid sequence selected from the group consisting of SEQ ID NO 1-8, or a homologue thereof having at least 66% sequence identity therewith.

4. The binder-toxin fusion protein according to any one of the aforementioned claims, wherein the protein binder is selected from the group consisting of
an antibody
an antibody fragment or derivative retaining target binding capacity, or
an antibody mimetic.

5. The binder-toxin fusion protein according to any one of the aforementioned claims, wherein the fusion protein comprises a peptide linker connecting the binder, or a fragment thereof, with the toxin, or with a cleavable domain comprised in the toxin.

6. The binder-toxin fusion protein according to any one of the aforementioned claims, wherein
the peptide linker or the cleavable domain is specifically or non-specifically cleavable by an enzyme expressed by a mammalian cell, or an enzyme that is produced by a mammalian host,
the peptide linker or the cleavable domain is not cleavable by an enzyme expressed by a plant cell, or an enzyme that is produced by a plant host, or
the binder-toxin fusion protein is expressed in a transfected plant cell or transfected plant host.

7. The binder-toxin fusion protein according to any one of the aforementioned claims, wherein the protein binder binds to human CD20 or human CD79B.

8. The binder-toxin fusion protein according to any one of the aforementioned claims, wherein the binder-toxin fusion protein is one of the formats selected from the group consisting of
(scFv-Fc)-(linker)-toxin (dimer)
toxin-(linker)-Fc-VH/VL
tetramer of two HC and two LC-(linker)-toxin (IgG format)
tetramer of two LC and two HC-(linker)-toxin (IgG format), or
tetramer of two LC-(linker)-toxin and two HC-(linker)-toxin (IgG format) wherein the linker is optional.

9. The binder-toxin fusion protein according to any one of the aforementioned claims, wherein the plant or plant cell is from the genus Nicotiana.

10. The binder-toxin fusion protein according to any one of the aforementioned claims, wherein the cleavable linker or the cleavable domain in the protoxin comprises at least one cleavage site selected from the group consisting of
a) Endosomal and/or Lysosomal proteases cleavage site
b) Cytosolic protease cleavage site, and/or
c) Cell surface proteases cleavage site.

11. The binder-toxin fusion protein according to any one of the aforementioned claims, which protein comprises at least one plant-specific N-glycan.

12. A pharmaceutical composition comprising at least the binder-toxin fusion protein according to any one of the aforementioned claims, and optionally one or more pharmaceutically acceptable excipients.

13. A combination comprising (i) the binder-toxin fusion protein according to any one of claims 1-11 or the pharmaceutical composition according to claim 12, and (ii) one or more further therapeutically active compounds.

14. The binder-toxin fusion protein according to any one of claims 1-11, or the composition of claim 12, or the combination of claim 13, for (the manufacture of a medicament for) use in the treatment of a human or animal subject
suffering from,
being at risk of developing, and/or
being diagnosed for,
developing a neoplastic disease, or for the prevention of such condition.

15. A method for treating a human or animal subject
suffering from,
being at risk of developing, and/or
being diagnosed for
developing a neoplastic disease, or for the prevention of such condition, said method comprising the administration of a therapeutically effective amount of the binder-toxin fusion protein according to any one of claims 1-11, or the composition of claim 12, or the combination of claim 13.
